(19) United States
(12) Patent Application Publication
(10) Pub. No.: US 2025/0296910 A1
(43) Pub. Date: Sep. 25, 2025

(54) BENZAMIDE ENAMINONE DERIVATIVES AND METHODS OF USE THEREOF

(71) Applicant: University of Maryland, Eastern Shore, Princess Anne, MD (US)

(72) Inventors: Patrice L. Jackson-Ayotunde, Salisbury, MD (US); Isis Jemiyotan Amaye, Pittsburg, PA (US); Rhashanda D. Haywood, Princess Anne, MD (US)

(21) Appl. No.: 18/852,345

(22) PCT Filed: Mar. 27, 2023
(86) PCT No.: PCT/US23/64976

§ 371 Date: Sep. 27, 2024

Related U.S. Application Data
(60) Provisional application No. 63/324,579, filed on Mar. 28, 2022.

(51) Int. Cl. C07C 233/85; A61K 31/166

(52) U.S. Cl. CPC C07C 233/85; A61K 31/166

(57) ABSTRACT

The present disclosure encompasses novel benzamide enaminone compounds and pharmaceutically acceptable salts thereof and compositions including such compounds and pharmaceutically acceptable salts. The disclosure further encompasses methods of treating a subject at risk of having a seizure and/or methods of reducing or preventing the occurrence or severity of a seizure or seizure disorder associated therewith by administering to a subject in need thereof a compound or a pharmaceutically acceptable salt thereof or a composition thereof. In some embodiments, the disclosure encompasses compositions and combinations of agents that act synergistically to treat, prevent, or inhibit the occurrence of a seizure or a seizure disorder.

### CROSS-REFERENCE TO RELATED APPLICATIONS

The application claims the benefit of and priority to U.S. provisional patent application No. 63/324,579, which was filed on Mar. 28, 2022, the contents of which are incorporated herein by reference in its entirety.

### FIELD OF THE INVENTION

The field of the invention relates generally to novel benzamide enaminone compounds, the preparation thereof, and the use thereof to, for example, to treat seizures or seizure disorders.

### BACKGROUND OF THE INVENTION

Epilepsy is the fourth most common neurological disorder and is a chronic and often progressive disease. It affects approximately 3 million people in the United States and 70 million people world-wide. It is known that about 1 in every 26 Americans will be diagnosed with epilepsy at some point in their lifetime. Epilepsy is characterized by brief spontaneous recurrent, convulsive, and non-convulsive seizures caused by the hyperactivity of neuronal discharges in the brain. Epileptic seizures are divided into two main categories, (a) generalized seizures and (b) focal seizures. Generalized seizures begin with the electrical neuronal discharge affecting the entire brain, whereas in focal seizures the abnormal neuronal discharge is localized to one area of the brain. The management of epilepsy can be challenging due to the limited understanding of the pathophysiology of the disorder. The goal for treatment of the epilepsies is complete seizure freedom without major drug-induced side effects. Despite the optimal use of available antiepileptic drugs (AEDs), 30-35% of patients are considered to have drug-resistant epilepsy (DRE). According to the International League Against Epilepsy, DRE is defined as “failure of a patient's seizures to respond to at least two antiepileptic medications that are appropriately chosen and used for an adequate period to achieve and maintain seizure freedom.” DRE is becoming more of an increasing burden in the epilepsy community with no known treatments. As a result, there is an increased demand for development of novel therapeutics for the management of DRE.

Due to the clinical need for therapies to address DRE, the current disclosure encompasses the design, synthesis, and evaluation of novel benzamide enaminones as potential anticonvulsant agents.

### SUMMARY OF THE INVENTION

The disclosure encompasses novel benzamide enaminone derivatives and pharmaceutically acceptable salts thereof and compositions including such compounds and pharmaceutically acceptable salts.

The disclosure also encompasses methods of treating or preventing a seizure or mitigating the occurrence of a seizure or seizure disorder or reducing the risk of the occurrence of a seizure in a subject in need thereof by administering a fluorinated benzamide enaminone derivative or a pharmaceutically acceptable salt disclosed herein, or a composition including such compound derivative or salt thereof.

The disclosure also encompasses methods of treating or preventing a seizure or mitigating the occurrence of a seizure or seizure disorder or reducing the risk of the occurrence of a seizure in a subject in need thereof by administering a chlorinated benzamide enaminone derivative or a pharmaceutically acceptable salt disclosed herein, or a composition including such compound derivative or salt thereof.

In some embodiments, the disclosure provides compounds, compositions comprising the compounds, and methods for using the compounds and compositions in the prophylaxis and/or treatment of seizures and related disorders.

In some embodiments, the disclosure provides novel fluorinated N-phenyl or N-benzyl enaminones. The compounds may be in the form of pharmaceutically acceptable salts of the compounds. The compositions include pharmaceutical formulations comprising one or combinations of the compounds and are suitable for administration to a subject (e.g., a mammal, preferably a human) in need thereof. In an embodiment, the disclosure provides a composition comprising one or more of the compounds and a pharmaceutically acceptable carrier.

In some embodiments, the disclosure provides novel chlorinated N-phenyl, 3-pyrindinyl, or N-benzyl enaminones. The compounds may be in the form of pharmaceutically acceptable salts of the compounds. The compositions include pharmaceutical formulations comprising one or combinations of the compounds and are suitable for administration to a subject (e.g., a mammal, preferably a human) in need thereof. In an embodiment, the disclosure provides a composition comprising one or more of the compounds and a pharmaceutically acceptable carrier.

In an embodiment, the compounds, compositions, and methods of the invention include compounds of formula (I) and formula (Ia):

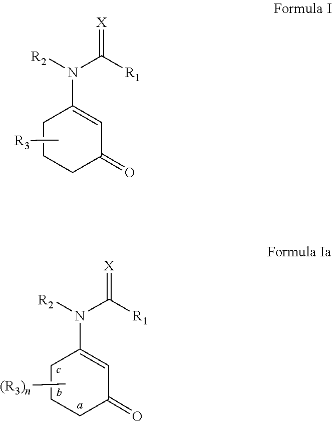

wherein
X is O or S;
R₁ is an aryl ring or heteroaryl ring with at least one halogen, alkyl, or alkyl substituted with 1-3 halogen groups, or R₁ is a substituted heteroaryl (e.g., substituted 3-pyridine);
R₂ is hydrogen or C₁-C₃ alkyl;
each R₃ is independently a hydroxyl, a halogen, cyano, amino, nitro, substituted or unsubstituted alkyl, substituted or unsubstituted alkenyl, substituted or unsubstituted alkynyl;
n is an integer from 1-6,
or a pharmaceutically acceptable salt, hydrate, solvate, enantiomer, diastereomer, racemate or mixture of stereoisomers thereof.

In certain embodiments, one or both of R₁ and R₃ include halogen substituents or a alkyl substituted with one or more halogen substituents.

In another embodiment, the compounds, compositions, and methods of the invention include compounds formula (II):

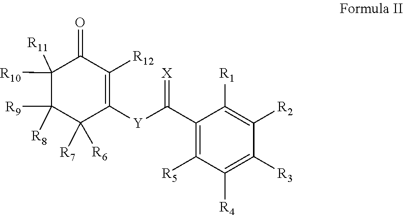

wherein
R₁ is —OH, —CN, substituted or unsubstituted thioalkyl, substituted or unsubstituted alkyl, substituted or unsubstituted alkenyl, substituted or unsubstituted alkynyl, halo, substituted or unsubstituted aryl, substituted or unsubstituted aryloxy, or substituted or unsubstituted alkyloxy;
each of R₂, R₃, R₄, or R₅ is independently selected from —H, —OH, —CN, substituted or unsubstituted thioalkyl, nitro, SCF₃, OCF₃, substituted or unsubstituted alkyl, substituted or unsubstituted alkenyl, substituted or unsubstituted alkynyl, halo, substituted or unsubstituted aryl, substituted or unsubstituted aryloxy, or substituted or unsubstituted alkyloxy;
each of R₆, R₇, R₈, R₉, R₁₀, or R₁₁ is independently selected from —H, —OH, —CN, substituted or unsubstituted thioalkyl, substituted or unsubstituted alkyl, substituted or unsubstituted alkenyl, substituted or unsubstituted alkynyl, halo, substituted or unsubstituted aryl, substituted or unsubstituted aryloxy, or substituted or unsubstituted alkyloxy;
R₁₂ is —H or substituted or unsubstituted alkyl;
X is O;
Y is —NR₁₃; and
each R₁₃ is independently selected from —H or substituted or unsubstituted alkyl (C1-C6 alkyl),
or a pharmaceutically acceptable salt, hydrate, solvate, enantiomer, diastereomer, racemate or mixture of stereoisomers thereof, wherein R₃ and R/R₉ are not identical.

In certain embodiments, at least one of R₁-R₅ is —F or —Cl, a substituted alkyl comprising one or more —F or —C1 groups, or a substituted alkyloxy comprising one or more —F groups.

In certain embodiments, at least one of R₇-R₁₂ is —F, a substituted alkyl comprising one or more —F groups, or a substituted alkyloxy comprising one or more —F groups.

In another embodiment, the compounds, compositions, and methods of the invention include compounds of formula (III) or formula (IIIa):

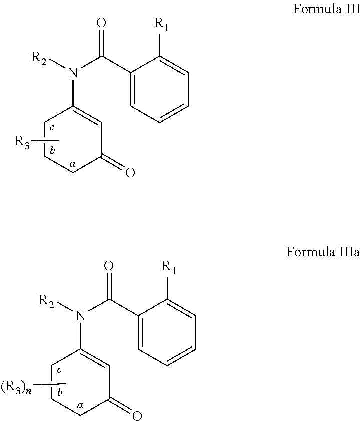

wherein
R₁ is a halogen or alkyl substituted with 1-3 halogen groups;
R₂ is hydrogen or C₁-C₃ alkyl;
R₃ is independently a hydroxyl, a halogen, cyano, amino, nitro, or alkyl substituted with 1-3 halogen groups;
n when present is an integer from 1 to 6;
or a pharmaceutically acceptable salt, hydrate, solvate, enantiomer, diastereomer, racemate or mixture of stereoisomers thereof.

In another embodiment, the compounds, compositions, and methods of the invention include compounds of formula (IV):

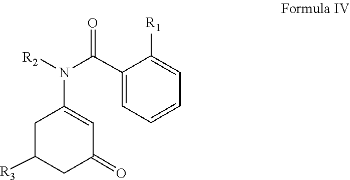

wherein
R₁ is a halogen or alkyl substituted with 1-3 halogen groups;
R₂ is hydrogen or C₁-C₃ alkyl; and
R₃ is a halogen or alkyl substituted with 1-3 halogen groups,
or a pharmaceutically acceptable salt, hydrate, solvate, enantiomer, diastereomer, racemate or mixture of stereoisomers thereof.

In certain embodiments, R₁ is a halogen comprising fluoride or chloride. In certain embodiments, R₁ is a fluoride.

In certain embodiments, R₁ is a substituted alkyl comprising one to three fluoride or chloride substituents. In certain embodiments, R₁ is trifluoromethyl.

The invention relates to methods of treating a subject at risk of having a seizure and/or methods of reducing or preventing the occurrence or severity of a seizure or seizure disorder associated therewith by administering to a subject in need thereof a therapeutically or prophylactically effective amount of a compound of Formula (I), (II), (III), or (IV) or a pharmaceutically acceptable salt thereof.

In various embodiments, administering a composition including a compound of Formula (I), (II), (III), or (IV) or a pharmaceutically acceptable salt or solvate thereof, to a subject in need thereof, results in one or more of the following: prevention of a subject from having a seizure; reduction in the duration or intensity of a seizure; reduction in the number of daily occurrences of a seizure; and or prevention or mitigation in the severity of a seizure.

The methods of the present invention provide advantages such as greater overall efficacy, for example, in achieving synergy or avoiding antagonism, and allow, where desired, a reduction in the amount of one or more of the individual agents employed with a concomitant reduction in side effects. Further, where the seizure to be treated is not optimally responsive to a given antiepileptic agent, use of the present pharmaceutical composition in the methods can nonetheless provide effective treatment.

The disclosure also includes pharmaceutical compositions comprising a compound of Formula (I), (II), (III), or (IV), and a pharmaceutically acceptable carrier or excipient. The pharmaceutical compositions contemplated to be within the scope of the invention, include pharmaceutically acceptable diluents, preservatives, solubilizers, emulsifiers, adjuvants and/or carriers. In various embodiments, the pharmaceutical compositions can be administered by any suitable route of administration, for example, parenterally, transmucosally, orally, nasally, or rectally, or transdermally. Preferably, administration is oral, e.g., via a tablet or capsule. Alternative means of administration also include, but are not limited to, intra-arteriole, intramuscular, intradermal, subcutaneous, intraperitoneal, intraventricular, and intracranial administration.

The pharmaceutical compositions of the invention can be delivered in a controlled release system, such as using an intravenous infusion, an implantable osmotic pump, a transdermal patch, or other modes of administration.

### BRIEF DESCRIPTION OF THE INVENTION

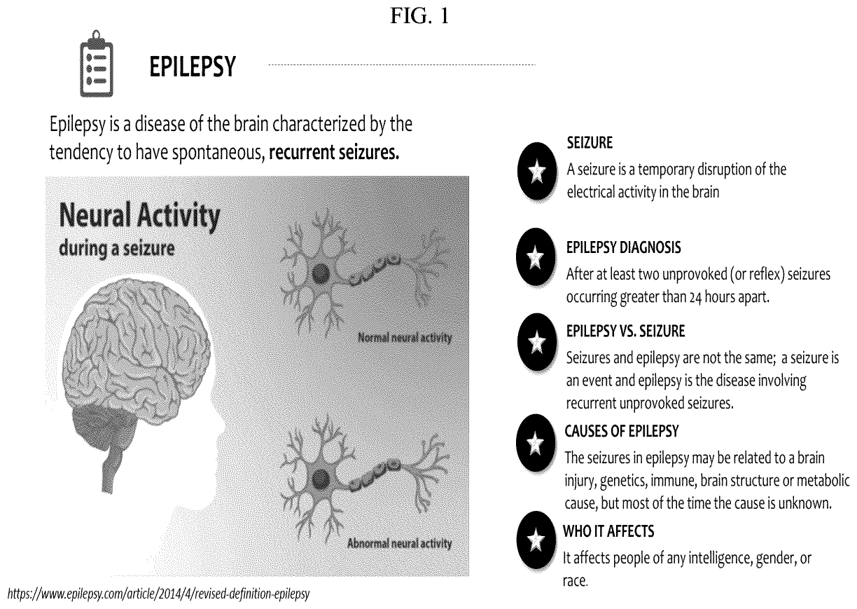

FIG. 1 illustrates an exemplary illustration of the neural activity during a seizure and characteristics of seizures.

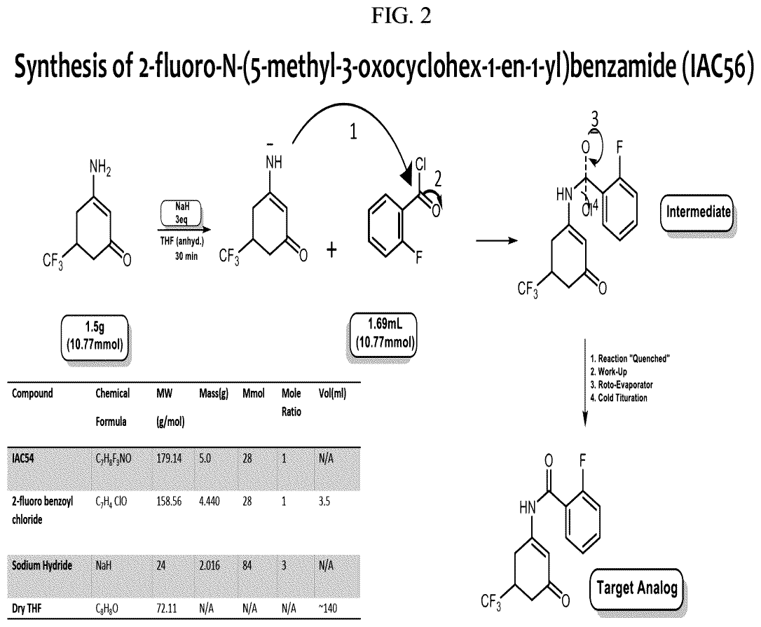

FIG. 2 illustrates an exemplary chemical synthesis of 2-fluoro-N-(5-methyl-3-oxocyclohex-1-en-1-yl)benzamide.

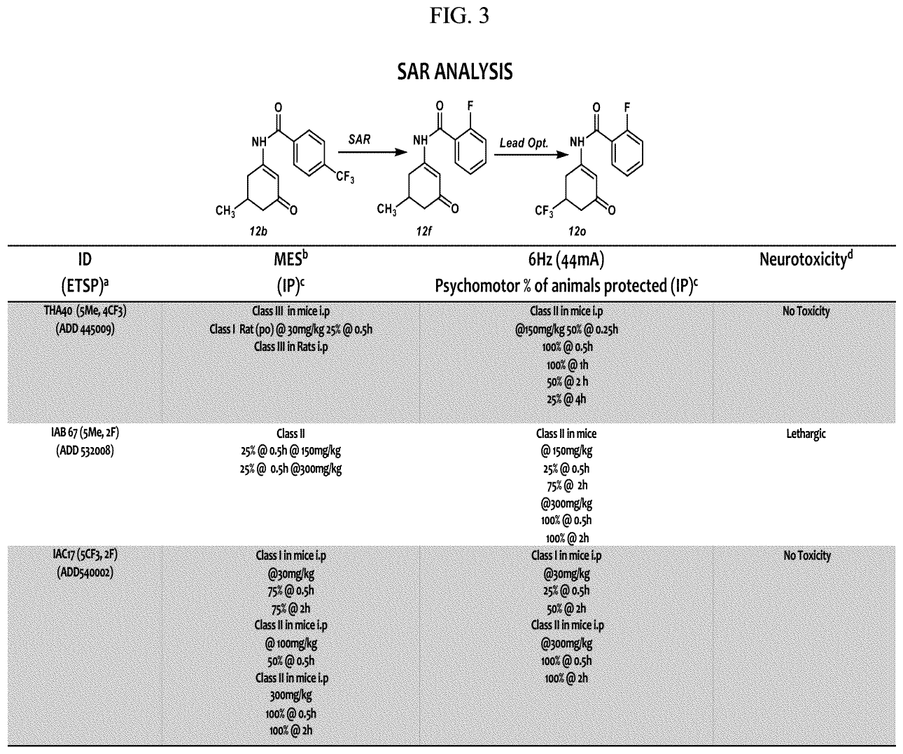

FIG. 3 illustrates structure activity studies for compounds during lead optimization.

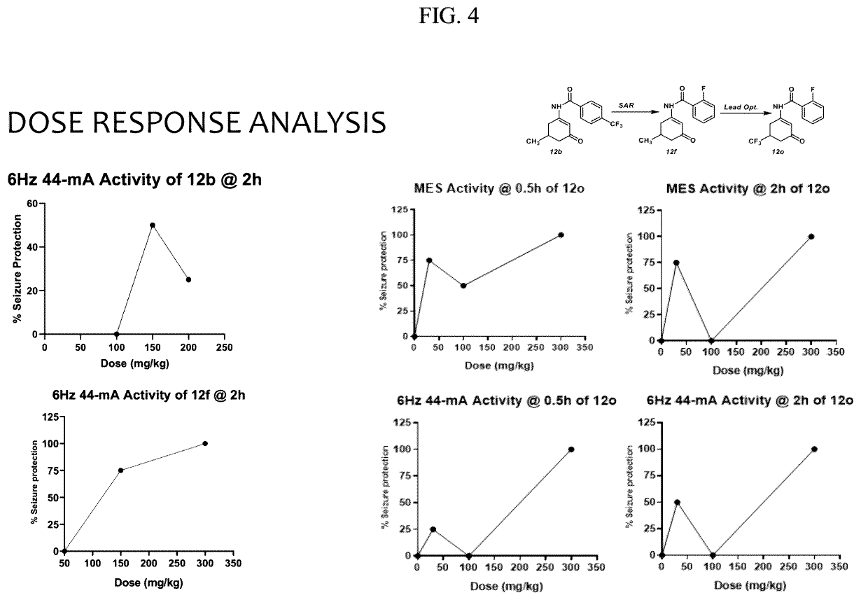

FIG. 4 illustrates dose response studies for lead compounds.

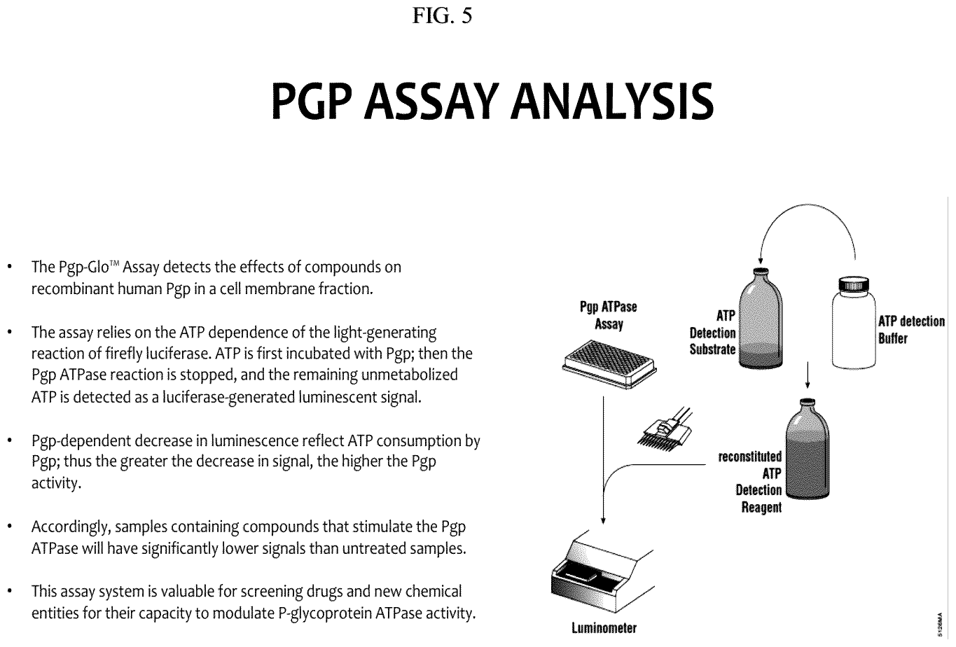

FIG. 5 illustrates a description of the PGP Assay Analysis.

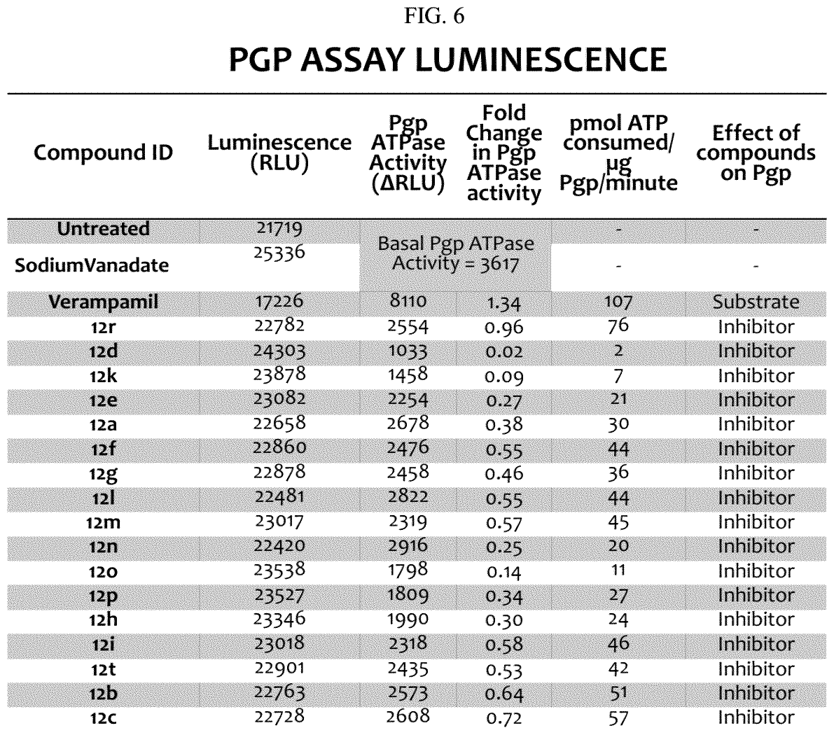

FIG. 6 illustrates PGP Assay luminescence for various exemplary compounds of the invention.

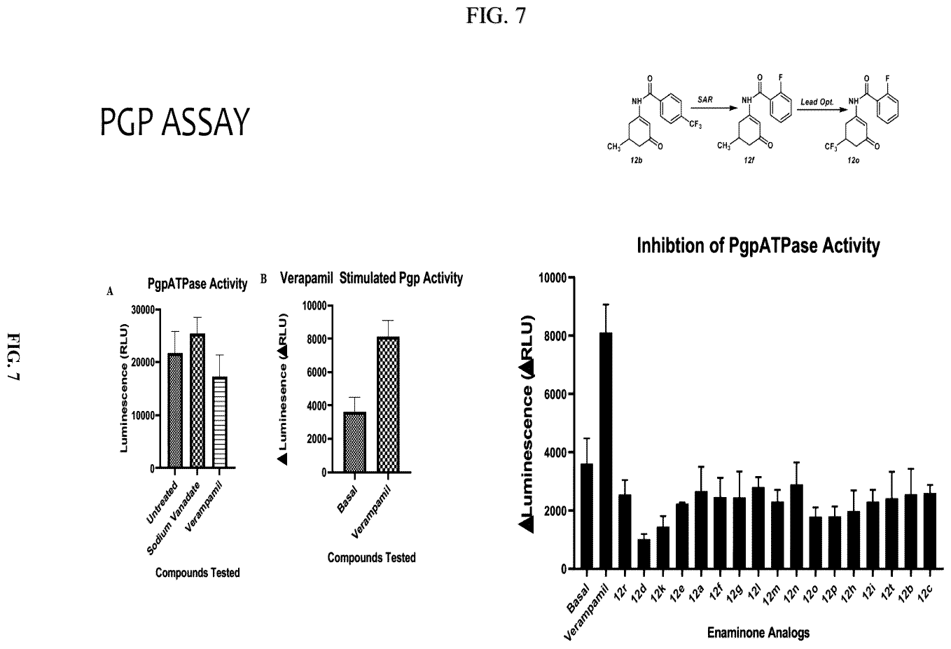

FIG. 7 illustrates PGP Assay results for various exemplary compounds of the invention.

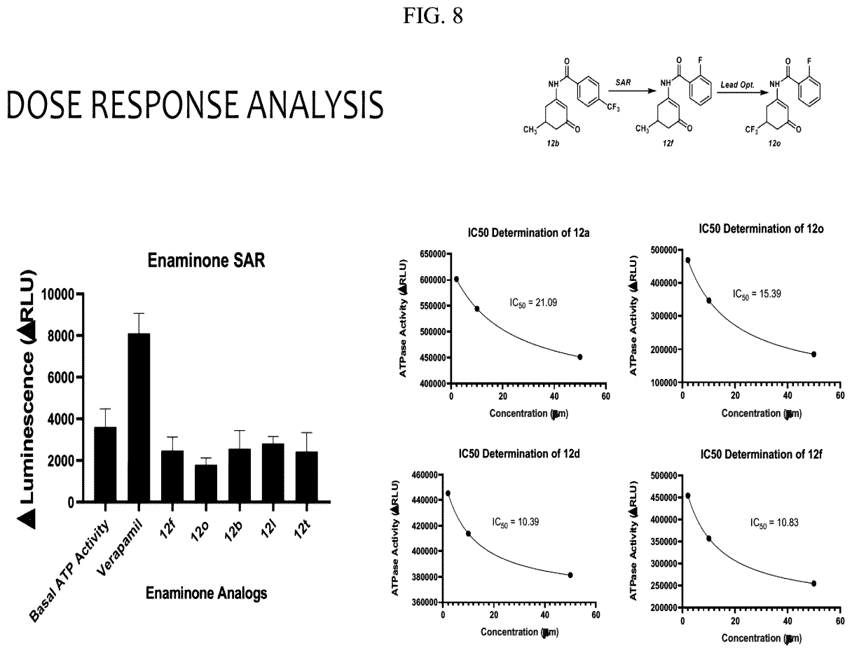

FIG. 8 illustrates does response analyses for various exemplary compounds of the invention.

### DETAILED DESCRIPTION OF THE INVENTION

### Definitions

Unless defined otherwise, all technical and scientific terms used herein have the same meanings as commonly understood by one of skill in the art to which the disclosure herein belongs.

The term “about” or “approximately” as used herein means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

As used herein, the term “alkyl” or “optionally substituted alkyl” refers to C₁-C₆ unsubstituted alkyl or alkyl having one or more substituents replacing one or more hydrogen atoms on one or more carbons of the hydrocarbon backbone. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkyl aminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

As used herein, the term “alkenyl” includes unsaturated aliphatic groups analogous in length and possible substitution to the alkyls described above, but that contain at least one double bond. For example, the term “alkenyl” includes straight chain alkenyl groups (e.g., ethenyl, propenyl, butenyl, pentenyl, hexenyl, heptenyl, octenyl, nonenyl, decenyl), and branched alkenyl groups. In certain embodiments, a straight chain or branched alkenyl group has six or fewer carbon atoms in its backbone (e.g., C₁-C₆ for straight chain, C₃-C₆ for branched chain). The term “C2-C₆” includes alkenyl groups containing two to six carbon atoms. The term “C₃-C₆” includes alkenyl groups containing three to six carbon atoms. The term “optionally substituted alkenyl” refers to unsubstituted alkenyl or alkenyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, aryl carbonyloxy, alkoxycarbonyloxy, aryloxycarbonyloxy, carboxylate, alkylcarbonyl, arylcarbonyl, alkoxycarbonyl, aminocarbonyl, alkyl aminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, heterocyclyl, alkylaryl, or an aromatic or heteroaromatic moiety.

As used herein, the term “alkynyl” includes unsaturated aliphatic groups analogous in length and possible substitution to the alkyls described above, but which contain at least one triple bond. For example, “alkynyl” includes straight chain alkynyl groups (e.g., ethynyl, propynyl, butynyl, pentynyl, hexynyl, heptynyl, octynyl, nonynyl, decynyl), and branched alkynyl groups. In certain embodiments, a straight chain or branched alkynyl group has six or fewer carbon atoms in its backbone (e.g., C2-C₆ for straight chain, C₃-C₆ for branched chain). The term “C2-C₆” includes alkynyl groups containing two to six carbon atoms. The term “C₃-C₆” includes alkynyl groups containing three to six carbon atoms. The term “optionally substituted alkynyl” refers to unsubstituted alkynyl or alkynyl having designated substituents replacing one or more hydrogen atoms on one or more hydrocarbon backbone carbon atoms. Such substituents can include, for example, alkyl, alkenyl, alkynyl, halogen, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkyl aminocarbonyl, dialkylaminocarbonyl, alkylthiocarbonyl, alkoxyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Other optionally substituted moieties (such as optionally substituted cycloalkyl, heterocycloalkyl, aryl, or heteroaryl) include both the unsubstituted moieties and the moieties having one or more of the designated substituents. For example, substituted heterocycloalkyl includes those substituted with one or more alkyl groups, such as 2,2,6,6-tetramethyl-piperidinyl and 2,2,6,6-tetramethyl-1,2,3,6-tetrahydropyridinyl.

As used herein, “amine” or “amino” refers to unsubstituted or substituted --NH.sub.2. “Alkylamino” includes groups of compounds wherein nitrogen of --NH.sub.2 is bound to at least one alkyl group. Examples of alkylamino groups include benzylamino, methylamino, ethylamino, phenethylamino, etc. “Dialkylamino” includes groups wherein the nitrogen of —NH₂ is bound to at least two additional alkyl groups. Examples of dialkylamino groups include, but are not limited to, dimethylamino and diethylamino. “Arylamino” and “diarylamino” include groups wherein the nitrogen is bound to at least one or two aryl groups, respectively. “Aminoaryl” and “aminoaryloxy” refer to aryl and aryloxy substituted with amino. “Alkylarylamino,” “alkylaminoaryl” or “arylaminoalkyl” refers to an amino group which is bound to at least one alkyl group and at least one aryl group. “Alkaminoalkyl” refers to an alkyl, alkenyl, or alkynyl group bound to a nitrogen atom which is also bound to an alkyl group. “Acylamino” includes groups wherein nitrogen is bound to an acyl group. Examples of acylamino include, but are not limited to, alkylcarbonylamino, arylcarbonylamino, carbamoyl and ureido groups.

The term “amide” or “aminocarboxy” includes compounds or moieties that contain a nitrogen atom that is bound to the carbon of a carbonyl or a thiocarbonyl group. The term includes “alkaminocarboxy” groups that include alkyl, alkenyl or alkynyl groups bound to an amino group which is bound to the carbon of a carbonyl or thiocarbonyl group. It also includes “arylaminocarboxy” groups that include aryl or heteroaryl moieties bound to an amino group that is bound to the carbon of a carbonyl or thiocarbonyl group. The terms “alkylaminocarboxy”, “alkenylaminocarboxy”, “alkynylaminocarboxy” and “arylaminocarboxy” include moieties wherein alkyl, alkenyl, alkynyl and aryl moieties, respectively, are bound to a nitrogen atom which is in turn bound to the carbon of a carbonyl group. Amides can be substituted with substituents such as straight chain alkyl, branched alkyl, cycloalkyl, aryl, heteroaryl or heterocycle. Substituents on amide groups may be further substituted.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound. “Analog” “analogue, and “derivative” are used herein interchangeably and refer to a compound that possesses the same core as the parent compound, but may differ from the parent compound in bond order, the absence or presence of one or more atoms and/or groups of atoms, and combinations thereof. The derivative can differ from the parent compound, for example, in one or more substituents present on the core, which may include one or more atoms, functional groups, or substructures. In general, a derivative can be imagined to be formed, at least theoretically, from the parent compound via chemical and/or physical processes.

As used herein, the term “aryl” includes groups with aromaticity, including “conjugated,” or multicyclic systems with at least one aromatic ring and do not contain any heteroatom in the ring structure. Examples include phenyl, benzyl, 1,2,3,4-tetrahydronaphthalenyl, etc. Furthermore, the terms “aryl” and “heteroaryl” include multicyclic aryl and heteroaryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, naphthrydine, indole, benzofuran, purine, benzofuran, deazapurine, indolizine. In the case of multicyclic aromatic rings, only one of the rings needs to be aromatic (e.g., 2,3-dihydroindole), although all of the rings may be aromatic (e.g., quinoline). The second ring can also be fused or bridged. The aryl, or heteroaryl ring can be substituted at one or more ring positions (e.g., the ring-forming carbon or heteroatom such as N) with such substituents as described above, for example, alkyl, alkenyl, alkynyl, halogen, hydroxyl, alkoxy, alkyl carbonyloxy, aryl carbonyloxy, alkoxycarbonyloxy, aryloxycarbonyloxy, carboxylate, alkylcarbonyl, alkylaminocarbonyl, aralkylaminocarbonyl, alkenylaminocarbonyl, alkylcarbonyl, arylcarbonyl, aralkylcarbonyl, alkenylcarbonyl, alkoxycarbonyl, aminocarbonyl, alkylthiocarbonyl, phosphate, phosphonato, phosphinato, amino (including alkylamino, dialkylamino, arylamino, diarylamino and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl and heteroaryl groups can also be fused or bridged with alicyclic or heterocyclic rings, which are not aromatic so as to form a multicyclic system (e.g., tetralin, methylenedioxyphenyl).

As used herein, the term “arylalkyl” or an “aralkyl” moiety is an alkyl substituted with an aryl (e.g., phenylmethyl (benzyl)). An “alkylaryl” moiety is an aryl substituted with an alkyl (e.g., methylphenyl).

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

The term “carbonyl” includes compounds and moieties which contain a carbon connected with a double bond to an oxygen atom. Examples of moieties containing a carbonyl include, but are not limited to, aldehydes, ketones, carboxylic acids, amides, esters, anhydrides, etc.

As used herein, the term “carboxyl” refers to —COOH or its C₁-C₆ alkyl ester.

As used herein, the compounds of the invention includes novel compounds encompassed by formulas (I), (II), (III), and (IV). The disclosure of compounds of formula (I), (II), (III), and/or (IV) includes compounds (I), (Ia), (II), (III), (IIIa), and (IV). The disclosure of compounds of formula (I) includes both compounds of formula (I) and formula (Ia). The disclosure of compounds of formula (III) includes both compounds of formula (III) and formula (IIIa).

As defined herein, the term “derivative” refers to compounds that have a common core structure, and are substituted with various groups as described herein. For example, all of the compounds represented by Formula (I), (II), (III), and (IV) are aryl- or heteroaryl-substituted benzene compounds, and have Formula (I), (II), (III), and (IV) as a common core.

As used herein, “dosage” is intended to encompass a formulation expressed in terms of μg/kg/day, μg/kg/hr, mg/kg/day or mg/kg/hr. The dosage is the amount of an ingredient administered in accordance with a particular dosage regimen. A “dose” is an amount of an agent administered to a mammal in a unit volume or mass, e.g., an absolute unit dose expressed in mg or μg of the agent. The dose depends on the concentration of the agent in the formulation, e.g., in moles per liter (M), mass per volume (m/v), or mass per mass (m/m). The two terms are closely related, as a particular dosage results from the regimen of administration of a dose or doses of the formulation. The particular meaning in any case will be apparent from context.

As used herein, the phrase “effective amount” or “therapeutically effective amount” of a compound or pharmaceutical composition refers to an amount sufficient to achieve the intended purpose, for example, preventing or reducing the number of seizures in a mammal, especially a human, including without limitation decreasing number or intensity of a seizure or preventing occurrence or duration of a seizure in an animal prior to administration, i.e., prophylactic administration. The terms also refer to an amount of a compound or salt thereof or composition thereof to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. In a preferred aspect, the disease or condition to be treated is a seizure or a seizure disorder.

The term “ester” includes compounds or moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc.

As used herein, “excipient” is a substance, other than the active drug substance, e.g., gaboxadol, of a pharmaceutical composition, which has been appropriately evaluated for safety and are included in a drug delivery system to either aid the processing of the drug delivery system during its manufacture; protect; support; enhance stability, bioavailability, or patient acceptability; assist in product identification; or enhance any other attributes of the overall safety and effectiveness of the drug delivery system during storage or use.

As used herein, “halo” or “halogen” refers to fluoro, chloro, bromo and iodo. The term “haloalkyl” or “haloalkoxyl” refers to an alkyl or alkoxyl substituted with one or more halogen atoms.

As used herein, the term “heteroaryl” groups are aryl groups, as defined above, except having from one to four heteroatoms in the ring structure, and may also be referred to as “aryl heterocycles” or “heteroaromatics.” As used herein, the term “heteroaryl” is intended to include a stable 5-, 6-, or 7-membered monocyclic or 7-, 8-, 9-, 10-, 11- or 12-membered bicyclic aromatic heterocyclic ring which consists of carbon atoms and one or more heteroatoms, e.g., 1 or 1-2 or 1-3 or 1-4 or 1-5 or 1-6 heteroatoms, or e.g., 1, 2, 3, 4, 5, or 6 heteroatoms, independently selected from the group consisting of nitrogen, oxygen and sulfur. The nitrogen atom may be substituted or unsubstituted (i.e., N or NR wherein R is H or other substituents, as defined). The nitrogen and sulfur heteroatoms may optionally be oxidized (i.e., N->O and S(O)ₚ, where p=1 or 2). It is to be noted that total number of S and O atoms in the aromatic heterocycle is not more than 1. Examples of heteroaryl groups include pyrrole, furan, thiophene, thiazole, isothiazole, imidazole, triazole, tetrazole, pyrazole, oxazole, isoxazole, pyridine, pyrazine, pyridazine, pyrimidine, and the like.

The term “hydroxy” or “hydroxyl” includes groups with an —OH.

As used herein, “improvement” refers to the treatment of symptoms or conditions associated with seizures or epileptic disorders, measured relative to at least one symptom or condition of the metabolic disease.

As used herein, “improvement in next day functioning” or “wherein there is improvement in next day functioning” refers to improvement after waking from an overnight sleep period wherein the beneficial effect of administration of a compound of formula (I), (II), or (III), or a pharmaceutically acceptable salt thereof, applies to at least one symptom or condition associated with an epileptic disorder and is discernable, either subjectively by a patient or objectively by an observer, for a period of time, e.g., immediately, 1 hour, 2 hours, hours, 3 hours, 4 hours, 5 hours, 6 hours, 12 hours, 24 hours, etc. after waking.

As used herein, the term “mitigate” or “mitigation” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as a seizure is alleviated if the severity or frequency of the seizure is reduced.

As used herein, “pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In embodiments, this term refers to molecular entities and compositions approved by a regulatory agency of the federal or a state government, as the GRAS list under section 204 (s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

As used herein, the term “prevention” or “preventing” means to administer a composition to a subject or a system at risk for or having a predisposition for one or more symptoms caused by a disease or disorder to facilitate cessation of a particular symptom of the disease or disorder, a reduction or prevention of one or more symptoms of the disease or disorder, a reduction in the severity of the disease or disorder, the complete ablation of the disease or disorder, stabilization or delay of the epileptic or progression of the disease or disorder.

As used herein, the term “seizures” and related “seizure disorders” that can treated, prevented or mitigated by administering a compound of the invention include, but are not limited to, epilepsy and related disorders and their attendant seizure symptoms. Non-limiting examples of seizure disorders include, but are not limited to, epilepsy (including, but not limited to, localization-related epilepsies, generalized epilepsies, epilepsies with both generalized and/or local seizures, and the like), seizures associated with Lennox-Gastaut syndrome, seizures as a complication of a disease or condition (such as seizures associated with encephalopathy, phenylketonuria, juvenile Gaucher's disease, Unvericht-Lundborg's progressive myoclonic epilepsy, stroke, head trauma, stress, hormonal changes, drug use or withdrawal, alcohol use or withdrawal, sleep deprivation, fever, infection, brain cancer, essential tremor syndrome and restless limb syndrome, and the like), and the like. In embodiments, the disorder is selected from epilepsy (regardless of type, underlying cause or origin), essential tremor syndrome, or restless limb syndrome. In embodiments, the seizure disorder is a disease or condition that is mediated by elevated persistent sodium current and/or other neural ionotropic abnormalities. As will be recognized in the art, a characteristic that distinguishes categories of seizures is whether the seizure activity is partial (e.g., focal) or generalized. In an embodiment, a compound/composition of the present disclosure is used to treat partial and/or generalized seizures. Partial seizures are considered those in which the seizure activity is restricted to discrete areas of the cerebral cortex. As is known in the art, if consciousness is fully preserved during the seizure, the seizure is considered to be a simple-partial seizure. If consciousness is impaired, the seizure is considered to be a complex-partial seizure. Within these types of seizures are included those that initiate as partial seizures and subsequently extend through the cortex; these are considered partial seizures with secondary generalization. Generalized seizures encompass distant regions of the brain simultaneously in a bilaterally symmetric manner and can include sudden, brief lapses of consciousness, such as in the case of absence or petit mal seizures, without loss of postural control. Atypical absence seizures usually include a longer period of lapse of consciousness, and more gradual onset and termination. Generalized tonic-clonic or grand mal seizures, which are considered to be the main type of generalized seizures, are characterized by abrupt onset, without warning. The initial phase of the seizure is usually tonic contraction of muscles, impaired respiration, a marked enhancement of sympathetic tone leading to increased heart rate, blood pressure, and pupillary size. After 10-20 seconds, the tonic phase of the seizure typically evolves into the clonic phase, produced by the superimposition of periods of muscle relaxation on the tonic muscle contraction. The periods of relaxation progressively increase until the end of the ictal phase, which usually lasts no more than one minute. The postictal phase is characterized by unresponsiveness, muscular flaccidity, and excessive salivation that can cause stridorous breathing and partial airway obstruction. Atonic seizures are characterized by sudden loss of postural muscle tone lasting 1-2 seconds. Consciousness is briefly impaired, but there is usually no postictal confusion. Myoclonic seizures are characterized by a sudden and brief muscle contraction that may involve one part of the body or the entire body. It is considered that the present disclosure is applicable for prophylaxis and/or therapy of any of the foregoing types of seizures, which are described for illustration but are not meant to be limiting. In embodiments, the disclosure is pertinent to treatment of epilepsy. In embodiments, the epilepsy is selected from idiopathic, cryptogenic, symptomatic, general and focal epilepsy. In embodiments, the disclosure is pertinent to treatment of pharmacoresistant epilepsy. As used herein, the term pharmacoresistant epilepsy means an epilepsy that is not controlled despite use of at least two drugs that are suitable for the type of epilepsy and have been appropriately prescribed at maximum tolerated doses. In embodiments the pharmacoresistant epilepsy is one where three such drugs trials have failed to eliminate the seizures. Those skilled in the art will recognize that the chances of controlling epilepsy decline sharply after failure of the second or third antiepileptic drug trial, and thus the present disclosure provides an approach designed to address these failed treatment cases.

As used herein, the term “severity” is meant to describe the potential of a seizure to be lessened or for example the reduction in the frequency of a seizure.

In the present specification, the structural formula of the compounds of Formula (I), (II), (III), or (IV) represent a certain isomer for convenience in some cases, but the present invention includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the present invention. Furthermore, so-called metabolite which is produced by degradation of the present compound in vivo is included in the scope of the present invention. As used herein, “stereoisomer,” refers to isomeric molecules that have the same molecular formula and sequence of bonded atoms (constitution), but which differ in the three dimensional orientations of their atoms in space. Examples of stereoisomers include enantiomers and diastereomers. As used herein, an enantiomer refers to one of the two mirror-image forms of an optically active or chiral molecule. Diastereomers (or diastereoisomers) are stereoisomers that are not enantiomers (non-superimposable mirror images of each other). Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2n, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Enantiomers and/or diastereomers can be resolved or separated using techniques known in the art. “Chirality” also includes axial and planar chirality. It is to be understood that the structures and other compounds discussed in this invention include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques, it has been possible to separate mixtures of two atropic isomers in select cases.

As used herein, a “subject in need thereof” is a subject having seizures or a seizure related disorder, or a subject having an increased risk of developing such disorder relative to the population at large. A subject in need thereof can have a condition that predisposed them to a seizure. A “subject” includes a mammal. The mammal can be e.g., any mammal, e.g., a human, primate, bird, mouse, rat, fowl, dog, cat, cow, horse, goat, camel, sheep or a pig. Preferably, the mammal is a human. The subject of the present invention includes any human subject who has been diagnosed with, has symptoms of, or is at risk of developing seizure or a seizure disorder.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

The compounds of Formula (I), (II), (III), and (IV) disclosed herein include the compounds themselves, as well as their salts, their esters, their solvates, and their prodrugs, if applicable. A salt, for example, can be formed between an anion and a positively charged group (e.g., amino) on an aryl- or heteroaryl-substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on an aryl- or heteroaryl-substituted benzene compound. Suitable cations include sodium ion, potassium ion, magnesium ion, calcium ion, and an ammonium cation such as tetramethylammonium ion. The aryl- or heteroaryl-substituted benzene compounds also include those salts containing quaternary nitrogen atoms. In the salt form, it is understood that the ratio of the compound to the cation or anion of the salt can be 1:1, or any ration other than 1:1, e.g., 3:1, 2:1, 1:2, or 1:3.

Additionally, the compounds of the invention, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “treating” or “treatment” refers to alleviating, attenuating or delaying the appearance of clinical symptoms of a disease or condition in a subject that may be afflicted with or predisposed to the disease or condition, but does not yet experience or display clinical or subclinical symptoms of the disease or condition. In certain embodiments, treating” or “treatment” may refer to preventing the appearance of clinical symptoms of a disease or condition in a subject that may be afflicted with or predisposed to the disease or condition, but does not yet experience or display clinical or subclinical symptoms of the disease or condition. “Treating” or “treatment” may also refer to inhibiting the disease or condition, e.g., arresting or reducing its epileptic or at least one clinical or subclinical symptom thereof “Treating” or “treatment” further refers to relieving the disease or condition, e.g., causing regression of the disease or condition or at least one of its clinical or subclinical symptoms. The benefit to a subject to be treated may be statistically significant, mathematically significant, or at least perceptible to the subject and/or the physician. Nonetheless, prophylactic (preventive) and therapeutic treatment are two separate embodiments of the disclosure herein. The inhibition is a measurable inhibition compared to a suitable control. In one embodiment, inhibition is at least 10 percent inhibition compared to a suitable control. That is, the rate of enzymatic activity or the amount of product with the inhibitor is less than or equal to 90 percent of the corresponding rate or amount made without the inhibitor. In various other embodiments, inhibition is at least 20, 25, 30, 40, 50, 60, 70, 75, 80, 90, or 95 percent inhibition compared to a suitable control. In one embodiment, inhibition is at least 99 percent inhibition compared to a suitable control. That is, the rate of enzymatic activity or the amount of product with the inhibitor is less than or equal to 1 percent of the corresponding rate or amount made without the inhibitor.

### Compounds and Compositions of the Invention

The disclosure also provides pharmaceutical compositions comprising a compound of Formula (I), (II), (III), or (IV) or pharmaceutically acceptable salts thereof mixed with pharmaceutically suitable carriers or excipient(s) at doses to treat or prevent a disease or condition (e.g., related to a seizure) as described herein.

In one embodiment, the compounds, compositions, and methods of the invention include compounds formula (I):

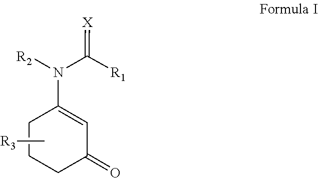

wherein
X is O or S;
R₁ is an aryl ring with at least one halogen, alkyl, or alkyl substituted with 1-3 halogen groups, or R₁ is a substituted heteroaryl (e.g., substituted 3-pyridine);
R₂ is hydrogen or C₁-C₃ alkyl;
R₃ is a hydroxyl, a halogen, cyano, amino, nitro, substituted or unsubstituted alkyl, substituted or unsubstituted alkenyl, substituted or unsubstituted alkynyl;
or a pharmaceutically acceptable salt, hydrate, solvate, enantiomer, diastereomer, racemate or mixture of stereoisomers thereof.

In certain embodiments, X is O.

In certain embodiments, X is S.

In certain embodiments, R₁ is an aryl ring with at least one halogen. In certain embodiments, the halogen is —F. In certain embodiments, the halogen is —Cl. In certain embodiments, R₁ is a phenyl ring with one —F. In certain embodiments, R₁ is a phenyl ring with one —Cl.

In certain embodiments, R₁ is an aryl ring with at least one alkyl group substituted with 1-3 halogen groups.

In certain embodiments, R₁ is an aryl ring with at least one alkyl group substituted with 1 halogen group.

In certain embodiments, R₁ is an aryl ring with at least one alkyl group substituted with 2 halogen groups.

In certain embodiments, R₁ is an aryl ring with at least one alkyl group substituted with 3 halogen groups.

In certain embodiments, R₁ is an aryl ring with at least one —CH₂F.

In certain embodiments, R₁ is an aryl ring with at least one —CHF₂.

In certain embodiments, R₁ is an aryl ring with at least one —CF₃.

In certain embodiments, R₁ is an aryl ring with at least one alkyl group.

In certain embodiments, R₁ is an aryl ring with at least one —CH₃.

In certain embodiments, R₁ is an aryl ring with at least one —CH₂CH₃.

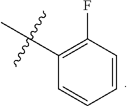 [SMILES: *c1ccccc1F]

In certain embodiments, R₁ is

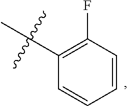 [SMILES: *c1ccccc1F]

In certain embodiments, when R₁ is R₃ is a C1-C₃-alkyl group substituted with 1-3 halogen groups (e.g., CF₃).

In certain embodiments, R₁ is

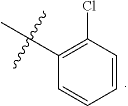 [SMILES: *c1ccccc1Cl]

In certain embodiments, R₁ is

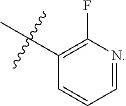 [SMILES: *c1cccnc1F]

In certain embodiments, R₁ is

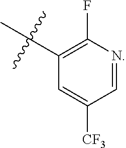 [SMILES: *c1cc(C(F)(F)F)cnc1F]

In certain embodiments, R₁ is

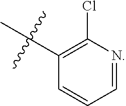 [SMILES: *c1cccnc1Cl]

In certain embodiments, R₁ is

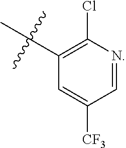 [SMILES: *c1cc(C(F)(F)F)cnc1Cl]

In certain embodiments, when R₁ is

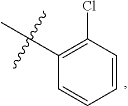 [SMILES: *c1ccccc1Cl]

R₃ is a C₁-C₆ alky (e.g., CHs), or a C₁-C₃-alkyl group substituted with 1-3 halogen groups (e.g., CF₃).

In certain embodiments, R₁ is

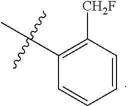

In certain embodiments, R₁ is

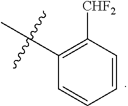 [SMILES: *c1ccccc1C(F)F]

In certain embodiments, R₁ is

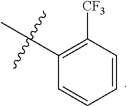 [SMILES: *c1ccccc1C(F)(F)F]

In certain embodiments, R₂ is hydrogen.

In certain embodiments, R₂ is C₁-C₃ alkyl.

In certain embodiments, R₂ is methyl.

In certain embodiments, R₂ is ethyl.

In certain embodiments, R₂ is propyl.

In certain embodiments, R₃ is a hydroxyl.

In certain embodiments, R₃ is a halogen.

In certain embodiments, R₃ is a cyano.

In certain embodiments, R₃ is an amino.

In certain embodiments, R₃ is a nitro.

In certain embodiments, R₃ is a substituted alkyl.

In certain embodiments, R₃ is a unsubstituted alkyl.

In certain embodiments, R₃ is a substituted alkenyl.

In certain embodiments, R₃ is a unsubstituted alkenyl.

In certain embodiments, R₃ is a substituted alkynyl.

In certain embodiments, R₃ is a unsubstituted alkynyl.

In certain embodiments, R₃ is an alkyl group substituted with 1 halogen group.

In certain embodiments, R₃ is an alkyl group substituted with 2 halogen groups.

In certain embodiments, R₃ is an alkyl group substituted with 3 halogen groups.

In certain embodiments, R₃ is —CH₂F.

In certain embodiments, R₃ is —CHF₂.

In certain embodiments, R₃ is —CF₃.

In certain embodiments, R₁ and R₃ include halogen substituents or a alkyl substituted with one or more halogen substituents.

In one embodiment, the compounds, compositions, and methods of the invention include compounds formula (Ia):

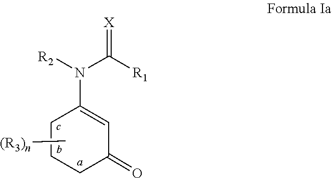

wherein
X is O or S;
R₁ is an aryl ring with at least one halogen, alkyl, or alkyl substituted with 1-3 halogen groups;
R₂ is hydrogen or C₁-C₃ alkyl;
R₃ is a hydroxyl, a halogen, cyano, amino, nitro, substituted or unsubstituted alkyl, substituted or unsubstituted alkenyl, substituted or unsubstituted alkynyl;
n is an integer from 1 to 6 (wherein positions a, b, and/or c can each contain 1 or 2 R₃ groups);
or a pharmaceutically acceptable salt, hydrate, solvate, enantiomer, diastereomer, racemate or mixture of stereoisomers thereof.

In certain embodiments, X is O.

In certain embodiments, X is S.

In certain embodiments, R₁ is an aryl ring with at least one halogen. In certain embodiments, the halogen is —F. In certain embodiments, the halogen is —Cl. In certain embodiments, R₁ is —F.

In certain embodiments, R₁ is an aryl ring with at least one alkyl group.

In certain embodiments, R₁ is an aryl ring with at least one alkyl group substituted with 1-3 halogen groups.

In certain embodiments, R₁ is an aryl ring with at least one alkyl group substituted with 1 halogen group.

In certain embodiments, R₁ is an aryl ring with at least one alkyl group substituted with 2 halogen groups.

In certain embodiments, R₁ is an aryl ring with at least one alkyl group substituted with 3 halogen groups.

In certain embodiments, R₁ is an aryl ring with at least one —CH₂F.

In certain embodiments, R₁ is an aryl ring with at least one —CHF₂.

In certain embodiments, R₁ is an aryl ring with at least one-CF₃.

In certain embodiments, R₁ is

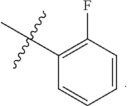 [SMILES: *c1ccccc1F]

In certain embodiments, R₁ is

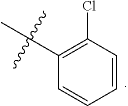 [SMILES: *c1ccccc1Cl]

In certain embodiments, R₁ is

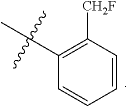

In certain embodiments, R₁ is

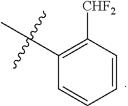 [SMILES: *c1ccccc1C(F)F]

In certain embodiments, R₁ is

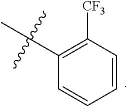 [SMILES: *c1ccccc1C(F)(F)F]

In certain embodiments, R₂ is hydrogen.

In certain embodiments, R₂ is C₁-C₃ alkyl.

In certain embodiments, R₂ is methyl.

In certain embodiments, R₂ is ethyl.

In certain embodiments, R₂ is propyl.

In certain embodiments, R₃ is a hydroxyl.

In certain embodiments, R₃ is a halogen.

In certain embodiments, R₃ is a cyano.

In certain embodiments, R₃ is an amino.

In certain embodiments, R₃ is a nitro.

In certain embodiments, R₃ is a substituted alkyl.

In certain embodiments, R₃ is a unsubstituted alkyl.

In certain embodiments, R₃ is a substituted alkenyl.

In certain embodiments, R₃ is a unsubstituted alkenyl.

In certain embodiments, R₃ is a substituted alkynyl.

In certain embodiments, R₃ is a unsubstituted alkynyl.

In certain embodiments, R₃ is an alkyl group substituted with 1 halogen group.

In certain embodiments, R₃ is an alkyl group substituted with 2 halogen groups.

In certain embodiments, R₃ is an alkyl group substituted with 3 halogen groups.

In certain embodiments, R₃ is —CH₂F.

In certain embodiments, R₃ is —CHF₂.

In certain embodiments, R₃ is —CF₃.

In certain embodiments, R₁ and R₃ include halogen substituents or a alkyl substituted with one or more halogen substituents.

In certain embodiments, n is 1.

In certain embodiments, n is 2.

In certain embodiments, n is 3.

In certain embodiments, n is 4.

In certain embodiments, n is 5.

In certain embodiments, n is 6.

In another embodiment, the compounds, compositions, and methods of the invention include compounds formula (II):

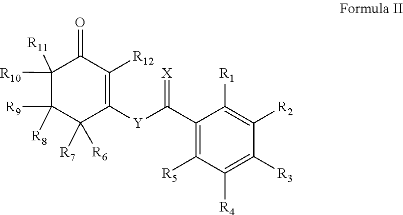

wherein
R₁ is hydrogen, —OH, —CN, substituted or unsubstituted thioalkyl, substituted or unsubstituted alkyl, substituted or unsubstituted alkenyl, substituted or unsubstituted alkynyl, halo, substituted or unsubstituted aryl, substituted or unsubstituted heteroaryl, substituted or unsubstituted aryloxy, or substituted or unsubstituted alkyloxy;
each of R₂, R₃, R₄, or R₅ is independently selected from —H, —OH, —CN, substituted or unsubstituted thioalkyl, substituted or unsubstituted alkyl, substituted or unsubstituted alkenyl, substituted or unsubstituted alkynyl, halo, substituted or unsubstituted aryl, substituted or unsubstituted heteroaryl, substituted or unsubstituted aryloxy, or substituted or unsubstituted alkyloxy, OCF₃. SCF₃, and NO₂;
each of R₆, R₇, R₈, R₉, R₁₀, or R₁₁ is independently selected from —H, —OH, —CN,
substituted or unsubstituted thioalkyl, substituted or unsubstituted alkyl, substituted or unsubstituted alkenyl, substituted or unsubstituted alkynyl, halo, substituted or unsubstituted aryl, substituted or unsubstituted heteroaryl, substituted or unsubstituted aryloxy, or substituted or unsubstituted alkyloxy;
R₁₂ is —H or substituted or unsubstituted alkyl;
X is O;
Y is —NR₁₃; and
each R₁₃ is independently selected from —H or substituted or unsubstituted alkyl (C1-C6 alkyl),
or a pharmaceutically acceptable salt, hydrate, solvate, enantiomer, diastereomer, racemate or mixture of stereoisomers thereof, wherein
at least one of R₁-R₅ is —F, a substituted alkyl comprising one or more —F groups, or a substituted alkyloxy comprising one or more —F groups, and
at least one of R₇-R₁₂ is —F, a substituted alkyl comprising one or more —F groups, or a substituted alkyloxy comprising one or more —F groups, and
wherein R₃ and R₈/R₉ are not identical.

In certain embodiments, R₁ is —H.

In certain embodiments, R₁ is —OH.

In certain embodiments, R₁ is —CN.

In certain embodiments, R₁ is substituted thioalkyl.

In certain embodiments, R₁ is unsubstituted thioalkyl.

In certain embodiments, R₁ is substituted alkyl.

In certain embodiments, R₁ is hydrogen.

In certain embodiments, R₁ is CH₂F.

In certain embodiments, R₁ is CHF₂.

In certain embodiments, R₁ is CF₃.

In certain embodiments, R₁ is unsubstituted alkyl.

In certain embodiments, R₁ is substituted alkenyl.

In certain embodiments, R₁ is unsubstituted alkenyl.

In certain embodiments, R₁ is substituted alkynyl.

In certain embodiments, R₁ is unsubstituted alkynyl.

In certain embodiments, R₁ is halo.

In certain embodiments, R₁ is —F.

In certain embodiments, R₁ is —Cl.

In certain embodiments, R₁ is substituted aryl.

In certain embodiments, R₁ is unsubstituted aryl.

In certain embodiments, R₁ is substituted heteroaryl.

In certain embodiments, R₁ is unsubstituted heteroaryl.

In certain embodiments, R₁ is substituted aryloxy.

In certain embodiments, R₁ is unsubstituted aryloxy.

In certain embodiments, R₁ is substituted alkyloxy.

In certain embodiments, R₁ is unsubstituted alkyloxy.

In certain embodiments, R₂ is —OH.

In certain embodiments, R₂ is —CN.

In certain embodiments, R₂ is substituted thioalkyl.

In certain embodiments, R₂ is unsubstituted thioalkyl.

In certain embodiments, R₂ is substituted alkyl.

In certain embodiments, R₂ is CH₂F.

In certain embodiments, R₂ is CHF₂.

In certain embodiments, R₂ is CF₃.

In certain embodiments, R₂ is unsubstituted alkyl.

In certain embodiments, R₂ is substituted alkenyl.

In certain embodiments, R₂ is unsubstituted alkenyl.

In certain embodiments, R₂ is substituted alkynyl.

In certain embodiments, R₂ is unsubstituted alkynyl.

In certain embodiments, R₂ is halo.

In certain embodiments, R₂ is —F.

In certain embodiments, R₂ is —Cl.

In certain embodiments, R₂ is substituted aryl.

In certain embodiments, R₂ is unsubstituted aryl.

In certain embodiments, R₂ is substituted heteroaryl.

In certain embodiments, R₂ is unsubstituted heteroaryl.

In certain embodiments, R₂ is substituted aryloxy.

In certain embodiments, R₂ is unsubstituted aryloxy.

In certain embodiments, R₂ is substituted alkyloxy.

In certain embodiments, R₂ is unsubstituted alkyloxy.

In certain embodiments, R₂ is —OCF₃.

In certain embodiments, R₂ is —SCF₃.

In certain embodiments, R₂ is —NO₂.

In certain embodiments, R₃ is —OH.

In certain embodiments, R₃ is —CN.

In certain embodiments, R₃ is substituted thioalkyl.

In certain embodiments, R₃ is unsubstituted thioalkyl.

In certain embodiments, R₃ is substituted alkyl.

In certain embodiments, R₃ is CH₂F.

In certain embodiments, R₃ is CHF₂.

In certain embodiments, R₃ is CF₃.

In certain embodiments, R₃ is unsubstituted alkyl.

In certain embodiments, R₃ is substituted alkenyl.

In certain embodiments, R₃ is unsubstituted alkenyl.

In certain embodiments, R₃ is substituted alkynyl.

In certain embodiments, R₃ is unsubstituted alkynyl.

In certain embodiments, R₃ is halo.

In certain embodiments, R₃ is —F.

In certain embodiments, R₃ is —Cl.

In certain embodiments, R₃ is substituted aryl.

In certain embodiments, R₃ is unsubstituted aryl.

In certain embodiments, R₃ is substituted heteroaryl.

In certain embodiments, R₃ is unsubstituted heteroaryl.

In certain embodiments, R₃ is substituted aryloxy.

In certain embodiments, R₃ is unsubstituted aryloxy.

In certain embodiments, R₃ is substituted alkyloxy.

In certain embodiments, R₃ is unsubstituted alkyloxy.

In certain embodiments, R₃ is —OCF₃.

In certain embodiments, R₃ is —SCF₃.

In certain embodiments, R₃ is —NO₂.

In certain embodiments, R₄ is —OH.

In certain embodiments, R₄ is —CN.

In certain embodiments, R₄ is substituted thioalkyl.

In certain embodiments, R₄ is unsubstituted thioalkyl.

In certain embodiments, R₄ is substituted alkyl.

In certain embodiments, R₄ is CH₂F.

In certain embodiments, R₄ is CHF₂.

In certain embodiments, R₄ is CF₃.

In certain embodiments, R₄ is unsubstituted alkyl.

In certain embodiments, R₄ is substituted alkenyl.

In certain embodiments, R₄ is unsubstituted alkenyl.

In certain embodiments, R₄ is substituted alkynyl.

In certain embodiments, R₄ is unsubstituted alkynyl.

In certain embodiments, R₄ is halo.

In certain embodiments, R₄ is —F.

In certain embodiments, R₄ is —Cl.

In certain embodiments, R₄ is substituted aryl.

In certain embodiments, R₄ is unsubstituted aryl.

In certain embodiments, R₄ is substituted heteroaryl.

In certain embodiments, R₄ is unsubstituted heteroaryl.

In certain embodiments, R₄ is substituted aryloxy.

In certain embodiments, R₄ is unsubstituted aryloxy.

In certain embodiments, R₄ is substituted alkyloxy.

In certain embodiments, R₄ is unsubstituted alkyloxy.

In certain embodiments, R₄ is —OCF₃.

In certain embodiments, R₄ is —SCF₃.

In certain embodiments, R₄ is —NO₂.

In certain embodiments, R₅ is —OH.

In certain embodiments, R₅ is —CN.

In certain embodiments, R₅ is substituted thioalkyl.

In certain embodiments, R₅ is unsubstituted thioalkyl.

In certain embodiments, R₅ is substituted alkyl.

In certain embodiments, R₅ is CH₂F.

In certain embodiments, R₅ is CHF₂.

In certain embodiments, R₅ is CF₃.

In certain embodiments, R₅ is unsubstituted alkyl.

In certain embodiments, R₅ is substituted alkenyl.

In certain embodiments, R₅ is unsubstituted alkenyl.

In certain embodiments, R₅ is substituted alkynyl.

In certain embodiments, R₅ is unsubstituted alkynyl.

In certain embodiments, R₅ is halo.

In certain embodiments, R₅ is —F.

In certain embodiments, R₅ is —Cl.

In certain embodiments, R₅ is substituted aryl.

In certain embodiments, R₅ is unsubstituted aryl.

In certain embodiments, R₅ is substituted heteroaryl.

In certain embodiments, R₅ is unsubstituted heteroaryl.

In certain embodiments, R₅ is substituted aryloxy.

In certain embodiments, R₅ is unsubstituted aryloxy.

In certain embodiments, R₅ is substituted alkyloxy.

In certain embodiments, R₅ is unsubstituted alkyloxy.

In certain embodiments, R₅ is —OCF₃.

In certain embodiments, R₅ is —SCF₃.

In certain embodiments, R₅ is —NO₂.

In certain embodiments, R₆ is —H.

In certain embodiments, R₆ is —OH.

In certain embodiments, R₆ is —CN.

In certain embodiments, R₆ is substituted thioalkyl.

In certain embodiments, R₆ is unsubstituted thioalkyl.

In certain embodiments, R₆ is substituted alkyl.

In certain embodiments, R₆ is CH₂F.

In certain embodiments, R₆ is CHF₂.

In certain embodiments, R₆ is CF₃.

In certain embodiments, R₆ is unsubstituted alkyl.

In certain embodiments, R₆ is substituted alkenyl.

In certain embodiments, R₆ is unsubstituted alkenyl.

In certain embodiments, R₆ is substituted alkynyl.

In certain embodiments, R₆ is unsubstituted alkynyl.

In certain embodiments, R₆ is halo.

In certain embodiments, R₆ is —F.

In certain embodiments, R₆ is —Cl.

In certain embodiments, R₆ is substituted aryl.

In certain embodiments, R₆ is unsubstituted aryl.

In certain embodiments, R₆ is substituted heteroaryl.

In certain embodiments, R₆ is unsubstituted heteroaryl.

In certain embodiments, R₆ is substituted aryloxy.

In certain embodiments, R₆ is unsubstituted aryloxy.

In certain embodiments, R₆ is substituted alkyloxy.

In certain embodiments, R₆ is unsubstituted alkyloxy.

In certain embodiments, R₇ is —H.

In certain embodiments, R₇ is —OH.

In certain embodiments, R₇ is —CN.

In certain embodiments, R₇ is substituted thioalkyl.

In certain embodiments, R₇ is unsubstituted thioalkyl.

In certain embodiments, R₇ is substituted alkyl.

In certain embodiments, R₇ is CH₂F.

In certain embodiments, R₇ is CHF₂.

In certain embodiments, R₇ is CF₃.

In certain embodiments, R₇ is unsubstituted alkyl.

In certain embodiments, R₇ is substituted alkenyl.

In certain embodiments, R₇ is unsubstituted alkenyl.

In certain embodiments, R₇ is substituted alkynyl.

In certain embodiments, R₇ is unsubstituted alkynyl.

In certain embodiments, R₇ is halo.

In certain embodiments, R₇ is —F.

In certain embodiments, R₇ is —Cl.

In certain embodiments, R₇ is substituted aryl.

In certain embodiments, R₇ is unsubstituted aryl.

In certain embodiments, R₇ is substituted heteroaryl.

In certain embodiments, R₇ is unsubstituted heteroaryl.

In certain embodiments, R₇ is substituted aryloxy.

In certain embodiments, R₇ is unsubstituted aryloxy.

In certain embodiments, R₇ is substituted alkyloxy.

In certain embodiments, R₇ is unsubstituted alkyloxy.

In certain embodiments, R₈ is —H.

In certain embodiments, R₈ is —OH.

In certain embodiments, R₈ is —CN.

In certain embodiments, R₈ is substituted thioalkyl.

In certain embodiments, R₈ is unsubstituted thioalkyl.

In certain embodiments, R₈ is substituted alkyl.

In certain embodiments, R₈ is CH₂F.

In certain embodiments, R₈ is CHF₂.

In certain embodiments, R₈ is CF₃.

In certain embodiments, R₈ is unsubstituted alkyl.

In certain embodiments, R₈ is substituted alkenyl.

In certain embodiments, R₈ is unsubstituted alkenyl.

In certain embodiments, R₈ is substituted alkynyl.

In certain embodiments, R₈ is unsubstituted alkynyl.

In certain embodiments, R₈ is halo.

In certain embodiments, R₈ is —F.

In certain embodiments, R₈ is —Cl.

In certain embodiments, R₈ is substituted aryl.

In certain embodiments, R₈ is unsubstituted aryl.

In certain embodiments, R₈ is substituted heteroaryl.

In certain embodiments, R₈ is unsubstituted heteroaryl.

In certain embodiments, R₈ is substituted aryloxy.

In certain embodiments, R₈ is unsubstituted aryloxy.

In certain embodiments, R₈ is substituted alkyloxy.

In certain embodiments, R₈ is unsubstituted alkyloxy.

In certain embodiments, R₉ is —H.

In certain embodiments, R₉ is —OH.

In certain embodiments, R₉ is —CN.

In certain embodiments, R₉ is substituted thioalkyl.

In certain embodiments, R₉ is unsubstituted thioalkyl.

In certain embodiments, R₉ is substituted alkyl.

In certain embodiments, R₉ is CH₂F.

In certain embodiments, R₉ is CHF₂.

In certain embodiments, R₉ is CF₃.

In certain embodiments, R₉ is unsubstituted alkyl.

In certain embodiments, R₉ is substituted alkenyl.

In certain embodiments, R₉ is unsubstituted alkenyl.

In certain embodiments, R₉ is substituted alkynyl.

In certain embodiments, R₉ is unsubstituted alkynyl.

In certain embodiments, R₉ is halo.

In certain embodiments, R₉ is —F.

In certain embodiments, R₉ is —Cl.

In certain embodiments, R₉ is substituted aryl.

In certain embodiments, R₉ is unsubstituted aryl.

In certain embodiments, R₉ is substituted heteroaryl.

In certain embodiments, R₉ is unsubstituted heteroaryl.

In certain embodiments, R₉ is substituted aryloxy.

In certain embodiments, R₉ is unsubstituted aryloxy.

In certain embodiments, R₉ is substituted alkyloxy.

In certain embodiments, R₉ is unsubstituted alkyloxy.

In certain embodiments, R₁₀ is —H.

In certain embodiments, R₁₀ is —OH.

In certain embodiments, R₁₀ is —CN.

In certain embodiments, R₁₀ is substituted thioalkyl.

In certain embodiments, R₁₀ is unsubstituted thioalkyl.

In certain embodiments, R₁₀ is substituted alkyl.

In certain embodiments, R₁₀ is CH₂F.

In certain embodiments, R₁₀ is CHF₂.

In certain embodiments, R₁₀ is CF₃.

In certain embodiments, R₁₀ is unsubstituted alkyl.

In certain embodiments, R₁₀ is substituted alkenyl.

In certain embodiments, R₁₀ is unsubstituted alkenyl.

In certain embodiments, R₁₀ is substituted alkynyl.

In certain embodiments, R₁₀ is unsubstituted alkynyl.

In certain embodiments, R₁₀ is halo.

In certain embodiments, R₁₀ is —F.

In certain embodiments, R₁₀ is —Cl.

In certain embodiments, R₁₀ is substituted aryl.

In certain embodiments, R₁₀ is unsubstituted aryl.

In certain embodiments, R₁₀ is substituted heteroaryl.

In certain embodiments, R₁₀ is unsubstituted heteroaryl.

In certain embodiments, R₁₀ is substituted aryloxy.

In certain embodiments, R₁₀ is unsubstituted aryloxy.

In certain embodiments, R₁₀ is substituted alkyloxy.

In certain embodiments, R₁₀ is unsubstituted alkyloxy.

In certain embodiments, R₁₁ is —H.

In certain embodiments, R₁₁ is —OH.

In certain embodiments, R₁₁ is —CN.

In certain embodiments, R₁₁ is substituted thioalkyl.

In certain embodiments, R₁₁ is unsubstituted thioalkyl.

In certain embodiments, R₁₁ is substituted alkyl.

In certain embodiments, R₁₁ is CH₂F.

In certain embodiments, R₁₁ is CHF₂.

In certain embodiments, R₁₁ is CF₃.

In certain embodiments, R₁₁ is unsubstituted alkyl.

In certain embodiments, R₁₁ is substituted alkenyl.

In certain embodiments, R₁₁ is unsubstituted alkenyl.

In certain embodiments, R₁₁ is substituted alkynyl.

In certain embodiments, R₁₁ is unsubstituted alkynyl.

In certain embodiments, R₁₁ is halo.

In certain embodiments, R₁₁ is —F.

In certain embodiments, R₁₁ is —Cl.

In certain embodiments, R₁₁ is substituted aryl.

In certain embodiments, R₁₁ is unsubstituted aryl.

In certain embodiments, R₁₁ is substituted heteroaryl.

In certain embodiments, Ru is unsubstituted heteroaryl.

In certain embodiments, Ru is substituted aryloxy.

In certain embodiments, R₁₁ is unsubstituted aryloxy.

In certain embodiments, R₁ is substituted alkyloxy.

In certain embodiments, R₁₁ is unsubstituted alkyloxy.

In certain embodiments, R₁₂ is —H.

In certain embodiments, R₁₂ is substituted alkyl.

In certain embodiments, R₁₂ is unsubstituted alkyl.

In certain embodiments, R₁₃ is —H.

In certain embodiments, R₁₃ is substituted alkyl.

In certain embodiments, R₁₃ is unsubstituted alkyl.

In certain embodiments, when R₁ is halogen or hydrogen, each of R₂, R₃, R₄, and R₅ is independently chosen from halogen (e.g. F or Cl), alkyl (e.g., CH₃), CF₃, alkoxy, OCF₃, CN, NO₂ and SCF₃.

In certain embodiments, when R₁ is halogen, each of R₂, R₃, R₄, and R₅ is hydrogen, R₆, R₇, R₁₀, R₁₁, and R₁₂ and either R₈ or R₉ is —CF₃.

In another embodiment, the compounds, compositions, and methods of the invention include compounds formula (III):

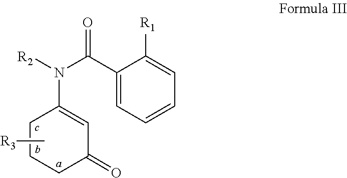

wherein
R₁ is a halogen or alkyl substituted with 1-3 halogen groups;
R₂ is hydrogen or C₁-C₃ alkyl;
R₃ is a hydroxyl, a halogen, cyano, amino, nitro, or alkyl substituted with 1-3 halogen groups, and one or more R₃ is at position a, b, or c or a combination thereof;
or a pharmaceutically acceptable salt, hydrate, solvate, enantiomer, diastereomer,
racemate or mixture of stereoisomers thereof.

In certain embodiments, R₁ is —F.

In certain embodiments, R₁ is —Cl.

In certain embodiments, R₁ is an alkyl group substituted with 1-3 halogen groups.

In certain embodiments, R₁ is an alkyl group substituted with 1 halogen group.

In certain embodiments, R₁ is an alkyl group substituted with 2 halogen groups.

In certain embodiments, R₁ is an alkyl group substituted with 3 halogen groups.

In certain embodiments, R₁ is —CH₂F.

In certain embodiments, R₁ is —CHF₂.

In certain embodiments, R₁ is —CF₃.

In certain embodiments, R₂ is hydrogen.

In certain embodiments, R₂ is C₁-C₃ alkyl.

In certain embodiments, R₂ is methyl.

In certain embodiments, R₂ is ethyl.

In certain embodiments, R₂ is propyl.

In certain embodiments, R₃ is a hydroxyl.

In certain embodiments, R₃ is a halogen.

In certain embodiments, R₃ is a cyano.

In certain embodiments, R₃ is an amino.

In certain embodiments, R₃ is a nitro.

In certain embodiments, R₃ is a substituted alkyl.

In certain embodiments, R₃ is a unsubstituted alkyl.

In certain embodiments, R₃ is a substituted alkenyl.

In certain embodiments, R₃ is a unsubstituted alkenyl.

In certain embodiments, R₃ is a substituted alkynyl.

In certain embodiments, R₃ is a unsubstituted alkynyl.

In certain embodiments, R₃ is an alkyl group substituted with 1 halogen group.

In certain embodiments, R₃ is an alkyl group substituted with 2 halogen groups.

In certain embodiments, R₃ is an alkyl group substituted with 3 halogen groups.

In certain embodiments, R₃ is —CH₂F.

In certain embodiments, R₃ is —CHF₂.

In certain embodiments, R₃ is —CF₃.

In certain embodiments, R₃ is at position a.

In certain embodiments, R₃ is at position b.

In certain embodiments, R₃ is at position c.

In certain embodiments, one R₃ is at position a and one R₃ is at position b.

In certain embodiments, one R₃ is at position a and one R₃ is at position c.

In certain embodiments, one R₃ is at position b and one R₃ is at position b.

In certain embodiments, one R₃ is at position a, one R₃ is at position b, and one R₃ is at position c.

In certain embodiments, R₁ and R₃ include halogen substituents or a alkyl substituted with one or more halogen substituents.

In certain embodiments, R₁ is —F and R₃ is —CF₃.

In another embodiment, the compounds, compositions, and methods of the invention include compounds formula (IIIa):

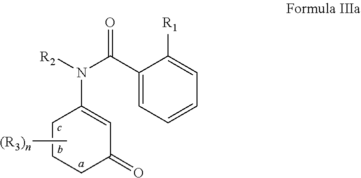

wherein
R₁ is a halogen or alkyl substituted with 1-3 halogen groups;
R₂ is hydrogen or C₁-C₃ alkyl;
each R₃ is independently a hydroxyl, a halogen, cyano, amino, nitro, or alkyl substituted with 1-3 halogen groups, and one or more R₃ is at position a, b, or c or a combination thereof;
n is an integer from 1 to 6 (wherein positions a, b, and/or c can each contain 1 or 2 R₃ groups);
or a pharmaceutically acceptable salt, hydrate, solvate, enantiomer, diastereomer, racemate or mixture of stereoisomers thereof.

In certain embodiments, R₁ is —F.

In certain embodiments, R₁ is —C1.

In certain embodiments, R₁ is an alkyl group substituted with 1-3 halogen groups.

In certain embodiments, R₁ is an alkyl group substituted with 1 halogen group.

In certain embodiments, R₁ is an alkyl group substituted with 2 halogen groups.

In certain embodiments, R₁ is an alkyl group substituted with 3 halogen groups.

In certain embodiments, R₁ is —CH₂F.

In certain embodiments, R₁ is —CHF₂.

In certain embodiments, R₁ is —CF₃.

In certain embodiments, R₂ is hydrogen.

In certain embodiments, R₂ is C₁-C₃ alkyl.

In certain embodiments, R₂ is methyl.

In certain embodiments, R₂ is ethyl.

In certain embodiments, R₂ is propyl.

In certain embodiments, R₃ is a hydroxyl.

In certain embodiments, R₃ is a halogen.

In certain embodiments, R₃ is a cyano.

In certain embodiments, R₃ is an amino.

In certain embodiments, R₃ is a nitro.

In certain embodiments, R₃ is a substituted alkyl.

In certain embodiments, R₃ is a unsubstituted alkyl.

In certain embodiments, R₃ is a substituted alkenyl.

In certain embodiments, R₃ is a unsubstituted alkenyl.

In certain embodiments, R₃ is a substituted alkynyl.

In certain embodiments, R₃ is a unsubstituted alkynyl.

In certain embodiments, R₃ is an alkyl group substituted with 1 halogen group.

In certain embodiments, R₃ is an alkyl group substituted with 2 halogen groups.

In certain embodiments, R₃ is an alkyl group substituted with 3 halogen groups.

In certain embodiments, R₃ is —CH₂F.

In certain embodiments, R₃ is —CHF₂.

In certain embodiments, R₃ is —CF₃.

In certain embodiments, R₃ is at position a.

In certain embodiments, R₃ is at position b.

In certain embodiments, R₃ is at position c.

In certain embodiments, one R₃ is at position a and one R₃ is at position b.

In certain embodiments, one R₃ is at position a and one R₃ is at position c.

In certain embodiments, one R₃ is at position b and one R₃ is at position b.

In certain embodiments, one R₃ is at position a, one R₃ is at position b, and one R₃ is at position c.

In certain embodiments, R₁ and R₃ include halogen substituents or a alkyl substituted with one or more halogen substituents.

In certain embodiments, R₁ is —F and R₃ is —CF₃.

In certain embodiments, n is 1.

In certain embodiments, n is 2.

In certain embodiments, n is 3.

In certain embodiments, n is 4.

In certain embodiments, n is 5.

In certain embodiments, n is 6.

In another embodiment, the compounds, compositions, and methods of the invention include compounds formula (IV):

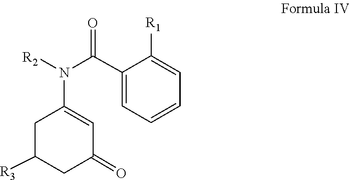

wherein
R₁ is a halogen, hydrogen, or alkyl substituted with 1-3 halogen groups;
R₂ is hydrogen or C₁-C₃ alkyl; and
R₃ is a halogen or alkyl substituted with 1-3 halogen groups,
or a pharmaceutically acceptable salt, hydrate, solvate, enantiomer, diastereomer, racemate or mixture of stereoisomers thereof.

In certain embodiments, R₁ is —F.

In certain embodiments, R₁ is —C1.

In certain embodiments, R₁ is an alkyl group substituted with 1-3 halogen groups.

In certain embodiments, R₁ is an alkyl group substituted with 1 halogen group.

In certain embodiments, R₁ is an alkyl group substituted with 2 halogen groups.

In certain embodiments, R₁ is an alkyl group substituted with 3 halogen groups.

In certain embodiments, R₁ is —CH₂F.

In certain embodiments, R₁ is —CHF₂.

In certain embodiments, R₁ is —CF₃.

In certain embodiments, R₂ is hydrogen.

In certain embodiments, R₂ is C₁-C₃ alkyl.

In certain embodiments, R₂ is methyl.

In certain embodiments, R₂ is ethyl.

In certain embodiments, R₂ is propyl.

In certain embodiments, R₃ is a hydroxyl.

In certain embodiments, R₃ is a halogen.

In certain embodiments, R₃ is a cyano.

In certain embodiments, R₃ is an amino.

In certain embodiments, R₃ is a nitro.

In certain embodiments, R₃ is a substituted alkyl.

In certain embodiments, R₃ is a unsubstituted alkyl.

In certain embodiments, R₃ is a substituted alkenyl.

In certain embodiments, R₃ is a unsubstituted alkenyl.

In certain embodiments, R₃ is a substituted alkynyl.

In certain embodiments, R₃ is a unsubstituted alkynyl.

In certain embodiments, R₅ is an alkyl group substituted with 1 halogen group.

In certain embodiments, R₃ is an alkyl group substituted with 2 halogen groups.

In certain embodiments, R₃ is an alkyl group substituted with 3 halogen groups.

In certain embodiments, R₃ is —CH₂F.

In certain embodiments, R₃ is —CHF₂.

In certain embodiments, R₃ is —CF₃.

Exemplary, non-limiting compounds of the invention are illustrated in the below table:

| COMPOUND | LAB ID | IUPAC |
|---|---|---|
| 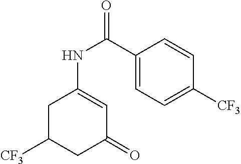 [SMILES: O=C1C=C(NC(=O)c2ccc(C(F)(F)F)cc2)CC(C(F)(F)F)C1] | IAC1 | N-(3-oxo-5- (trifluoromethyl) cyclohex- 1-en-1-yl)-4- (trifluoromethyl) benzamide |
| 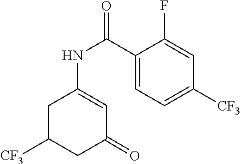 [SMILES: O=C1C=C(NC(=O)c2ccc(C(F)(F)F)cc2F)CC(C(F)(F)F)C1] | IAC5 | 2-fluoro-N-(3-oxo-5- (trifluoromethyl) cyclohex- 1-en-1-yl)-4- (trifluoromethyl) benzamide |
| 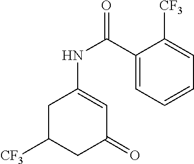 [SMILES: O=C1C=C(NC(=O)c2ccccc2C(F)(F)F)CC(C(F)(F)F)C1] | IAC9 | N-(3-oxo-5- (trifluoromethyl) cyclohex- 1-en-1-yl)-4- (trifluoromethyl) benzamide |
| 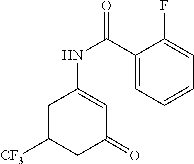 [SMILES: O=C1C=C(NC(=O)c2ccccc2F)CC(C(F)(F)F)C1] | IAC17 | 2-fluoro-N-(3-oxo-5- (trifluoromethyl) cyclohex- 1-en-1-yl) benzamide |
| 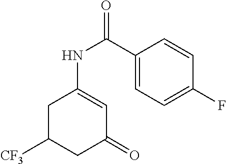 [SMILES: O=C1C=C(NC(=O)c2ccc(F)cc2)CC(C(F)(F)F)C1] | IAC46 | 4-fluoro-N-(3-oxo-5- (trifluoromethyl) cyclohex- 1-en-1-yl) benzamide |

Scheme I illustrates an exemplary synthetic methodology that may be used to develop meta substituted N-benzamide enaminone derivatives.

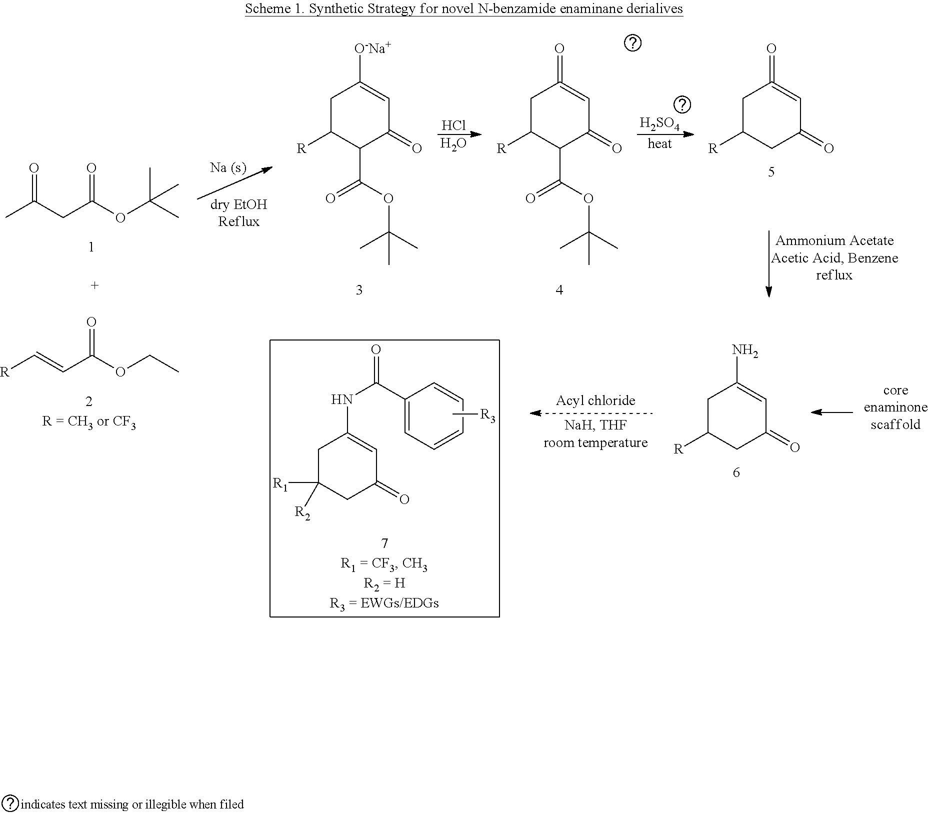

Exemplary, non-limiting compounds of the invention also include:

| | LAB | | |
|---|---|---|---|
| Compound Structure | ID | IUPAC Name | Mol Wt. |
| 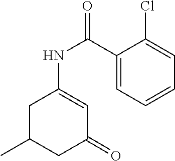 [SMILES: CC1CC(=O)C=C(NC(=O)c2ccccc2Cl)C1] | RHA- 88 | 2-chloro-N-(5-methyl-3- oxocyclohex-1-en-1- yl)benzamide | 263.72 g/mol |
| 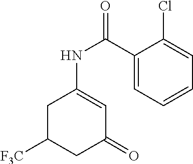 [SMILES: O=C1C=C(NC(=O)c2ccccc2Cl)CC(C(F)(F)F)C1] | RHB- 14 | 2-chloro-N-(3-oxo-5- (trifluoromethyl)cyclohex-1-en- 1-yl)benzamide | 317.69 g/mol |
| 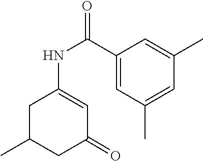 | RHB- 59 | 3,5-dimethyl-N-(5-methyl-3- oxocyclohex-1-en-1- yl)benzamide | 257.33 g/mol |
| 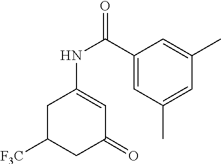 [SMILES: Cc1cc(C)cc(C(=O)NC2=CC(=O)CC(C(F)(F)F)C2)c1] | RHB- 62 | 3,5-dimethyl-N-(3-oxo-5- (trifluoromethyl)cyclohex-1-en- 1-yl)benzamide | 311.30 g/mol |
| 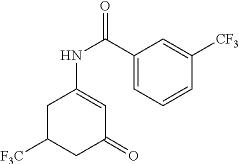 [SMILES: O=C1C=C(NC(=O)c2cccc(C(F)(F)F)c2)CC(C(F)(F)F)C1] | RHB- 95 | N-(3-oxo-5- (trifluoromethyl)cyclohex-1-en- 1-yl)-3- (trifluoromethyl)benzamide | 351.25 g/mol |
| 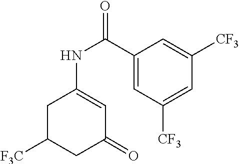 [SMILES: O=C1C=C(NC(=O)c2cc(C(F)(F)F)cc(C(F)(F)F)c2)CC(C(F)(F)F)C1] | RHB- 77 | N-(3-oxo-5- (trifluoromethyl)cyclohex-1-en- 1-yl)-3,5- bis(trifluoromethyl)benzamide | 419.25 g/mol |
| 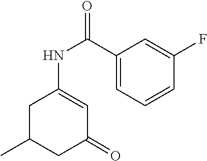 [SMILES: CC1CC(=O)C=C(NC(=O)c2cccc(F)c2)C1] | RHB- 145 | 3-fluoro-N-(5-methyl-3- oxocyclohex-1-en-1- yl)benzamide | 247.27 g/mol |
| 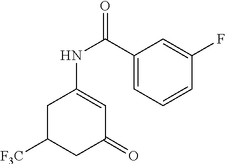 [SMILES: O=C1C=C(NC(=O)c2cccc(F)c2)CC(C(F)(F)F)C1] | RHB- 99 | 3-fluoro-N-(3-oxo-5- (trifluoromethyl)cyclohex-1-en- 1-yl)benzamide | 301.24 g/mol |
| 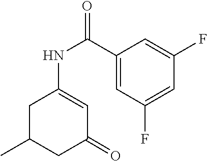 [SMILES: CC1CC(=O)C=C(NC(=O)c2cc(F)cc(F)c2)C1] | RHB- 109 | 3,5-difluoro-N-(5-methyl-3- oxocyclohex-1-en-1- yl)benzamide | 265.26 g/mol |
| 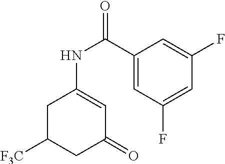 [SMILES: O=C1C=C(NC(=O)c2cc(F)cc(F)c2)CC(C(F)(F)F)C1] | RHB- 107 | 3,5-difluoro-N-(3-oxo-5- (trifluoromethyl)cyclohex-1-en- 1-yl)benzamide | 319.23 g/mol |
| 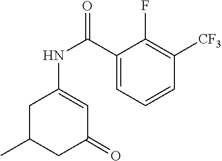 [SMILES: CC1CC(=O)C=C(NC(=O)c2cccc(C(F)(F)F)c2F)C1] | RHB- 111 | 2-fluoro-N-(5-methyl-3- oxocyclohex-1-en-1-yl)-3- (trifluoromethyl)benzamide | 315.27 g/mol |
| 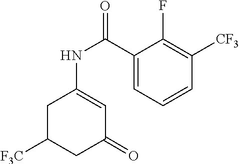 [SMILES: O=C1C=C(NC(=O)c2cccc(C(F)(F)F)c2F)CC(C(F)(F)F)C1] | RHB- 103 | 2-fluoro-N-(3-oxo-5- (trifluoromethyl)cyclohex-1-en- 1-yl)-3- (trifluoromethyl)benzamide | 369.24 g/mol |
| 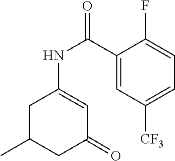 [SMILES: CC1CC(=O)C=C(NC(=O)c2cc(C(F)(F)F)ccc2F)C1] | RHB- 113 | 2-fluoro-N-(5-methyl-3- oxocyclohex-1-en-1-yl)-5- (trifluoromethyl)benzamide | 315.27 g/mol |
| 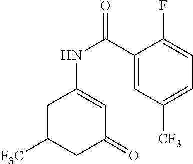 [SMILES: O=C1C=C(NC(=O)c2cc(C(F)(F)F)ccc2F)CC(C(F)(F)F)C1] | RHB- 115 | 2-fluoro-N-(3-oxo-5- (trifluoromethyl)cyclohex-1-en- 1-yl)-5- (trifluoromethyl)benzamide | 369.24 g/mol |
| 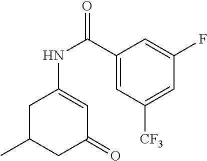 [SMILES: CC1CC(=O)C=C(NC(=O)c2cc(F)cc(C(F)(F)F)c2)C1] | RHB- 117 | 3-fluoro-N-(5-methyl-3- oxocyclohex-1-en-1-yl)-5- (trifluoromethyl)benzamide | 315.27 g/mol |
| 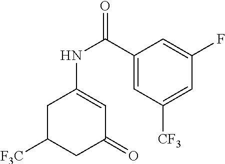 [SMILES: O=C1C=C(NC(=O)c2cc(F)cc(C(F)(F)F)c2)CC(C(F)(F)F)C1] | RHB- 119 | 3-fluoro-N-(3-oxo-5- (trifluoromethyl)cyclohex-1-en- 1-yl)-5- (trifluoromethyl)benzamide | 369.24 g/mol |
| 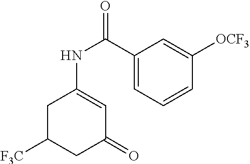 [SMILES: O=C1C=C(NC(=O)c2cccc(OC(F)(F)F)c2)CC(C(F)(F)F)C1] | RHB- 121 | N-(3-oxo-5- (trifluoromethyl)cyclohex-1-en- 1-yl)-3- (trifluoromethoxy)benzamide | 367.25 g/mol |
| 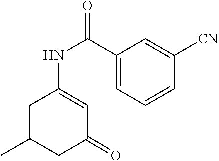 [SMILES: CC1CC(=O)C=C(NC(=O)c2cccc(C#N)c2)C1] | RHB- 123 | 3-cyano-N-(5-methyl-3- oxocyclohex-1-en-1- yl)benzamide | 254.29 g/mol |
| 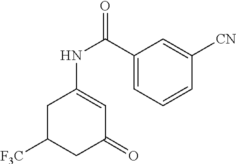 [SMILES: N#Cc1cccc(C(=O)NC2=CC(=O)CC(C(F)(F)F)C2)c1] | RHB- 125 | 3-cyano-N-(3-oxo-5- (trifluoromethyl)cyclohex-1-en- 1-yl)benzamide | 308.26 g/mol |
| 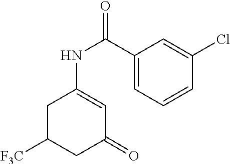 [SMILES: O=C1C=C(NC(=O)c2cccc(Cl)c2)CC(C(F)(F)F)C1] | RHB- 127 | 3-chloro-N-(3-oxo-5- (trifluoromethyl)cyclohex-1-en- 1-yl)benzamide | 317.69 g/mol |
| 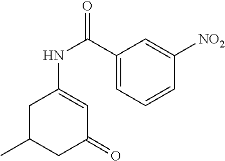 [SMILES: CC1CC(=O)C=C(NC(=O)c2cccc([N+](=O)[O-])c2)C1] | RHB- 129 | N-(5-methyl-3-oxocyclohex-1- en-1-yl)-3-nitrobenzamide | 274.28 g/mol |
| 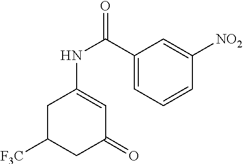 [SMILES: O=C1C=C(NC(=O)c2cccc([N+](=O)[O-])c2)CC(C(F)(F)F)C1] | RHB- 131 | 3-nitro-N-(3-oxo-5- (trifluoromethyl)cyclohex-1-en- 1-yl)benzamide | 328.25 g/mol |
| 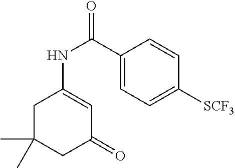 | RHB- 135 | N-(5,5-dimethyl-3-oxocyclohex- 1-en-1-yl)-4- ((trifluoromethyl)thio)benzamide | 343.36 g/mol |
| 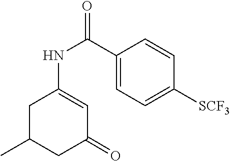 | RHB- 137 | N-(5-methyl-3-oxocyclohex-1- en-1-yl)-4- ((trifluoromethyl)thio)benzamide | 329.34 g/mol |
| 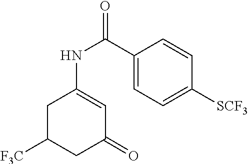 | RHB- 139 | N-(3-oxo-5- (trifluoromethyl)cyclohex-1-en- 1-yl)-4- ((trifluoromethyl)thio)benzamide | 383.31 g/mol |
| 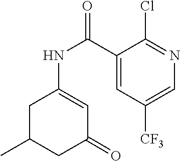 [SMILES: CC1CC(=O)C=C(NC(=O)c2cc(C(F)(F)F)cnc2Cl)C1] | RHB- 141 | 2-chloro-N-(5-methyl-3- oxocyclohex-1-en-1-yl)-5- (trifluoromethyl)nicotinamide | 332.71 g/mol |
| 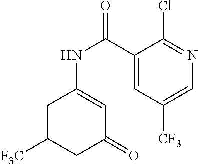 [SMILES: O=C1C=C(NC(=O)c2cc(C(F)(F)F)cnc2Cl)CC(C(F)(F)F)C1] | RHB- 143 | 2-chloro-N-(3-oxo-5- (trifluoromethyl)cyclohex-1-en- 1-yl)-5- (trifluoromethyl)nicotinamide | 386.68 g/mol |

In another embodiment, the disclosure also provides pharmaceutical compositions comprising compounds of Formula (I), (II), (III), or (IV) or pharmaceutically acceptable salts thereof, mixed with pharmaceutically suitable carriers or excipient(s) at doses to treat or prevent a disease or condition as described herein. The pharmaceutical compositions of the invention can also be administered in combination with other therapeutic agents or therapeutic modalities simultaneously, sequentially, or in alternation.

A composition of the invention comprises a compound of Formula (I), (II), (III), or (IV) or a pharmaceutically acceptable salt thereof. The present invention provides for the administration of a compound of Formula (I), (II), (III), or (IV) or a pharmaceutically acceptable salt thereof, and one or more additional therapeutic agents or a pharmaceutically acceptable salt thereof, as a co-formulation or separate formulations, wherein the administration of formulations is simultaneous, sequential, or in alternation. In certain embodiments, the other therapeutic agents can be an agent that is recognized in the art as being useful to treat the disease or condition being treated by the composition of the present invention. In other embodiment, the other therapeutic agent can be an agent that is not recognized in the art as being useful to treat the disease or condition being treated by the composition of the present invention. In one aspect, the other therapeutic agents can be an agent that imparts a beneficial attribute to the composition of the present invention (e.g., an agent that affects the viscosity of the composition). The beneficial attribute to the composition of the present invention includes, but is not limited to, pharmacokinetic or pharmacodynamic co-action resulting from the combination of a compound of Formula (I), (II), (III), or (IV) and one or more other therapeutic agents. For example, the one or more other therapeutic agents can be an antiepileptic drug.

Mixtures of compositions of the present invention can also be administered to the patient as a simple mixture or in suitable formulated pharmaceutical compositions. For example, one aspect of the invention relates to a pharmaceutical composition comprising a therapeutically effective dose of Formula (I), (II), (III), or (IV) or a pharmaceutically acceptable salt, enantiomer or stereoisomer thereof; one or more other therapeutic agents, and a pharmaceutically acceptable diluent or carrier.

A pharmaceutical composition is a formulation containing the compounds of Formula (I), (II), (III), or (IV) in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of humans and animals without excessive toxicity, irritation, allergic response, or other problem or complication, commensurate with a reasonable benefit/risk ratio.

Pharmaceutically acceptable excipient are excipients that are useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

A composition of the invention can be administered to a subject in many of the well-known methods currently used for chemotherapeutic treatment. For example, for treatment of the disorders disclosed herein, a compound of the invention may be injected into the blood stream or body cavities or taken orally or applied through the skin with patches. The dose chosen should be sufficient to constitute effective treatment but not so high as to cause unacceptable side effects. The state of the disease condition (e.g., seizures or seizure disorders, and the like) and the health of the patient should preferably be closely monitored during and for a reasonable period after treatment.

In certain embodiments, the therapeutically effective amount of each pharmaceutical agent used in combination will be lower when used in combination in comparison to monotherapy with each agent alone. Such lower therapeutically effective amount could afford for lower toxicity of the therapeutic regimen.

For any compound, the therapeutically effective amount can be estimated initially either in cell culture assays or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the compounds of formula (I), (II), (III), or (IV) or a pharmaceutically acceptable salt thereof to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing the compounds of formula (I), (II), (III), or (IV) or a pharmaceutically acceptable salt thereof may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL.TM. (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds of formula (I), (II), (III), or (IV) or a pharmaceutically acceptable salt thereof can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved.

In therapeutic applications, the dosages of the compounds of formula (I), (II), (III), or (IV) or a pharmaceutically acceptable salt thereof described herein, and optionally one or more other therapeutic agents, or the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages can range from about 0.001 mg/kg per day to about 5000 mg/kg per day. In preferred aspects, dosages can range from about 0.01 mg/kg per day to about 1000 mg/kg per day. In an aspect, the dose will be in the range of about 0.1 mg/day to about 50 g/day; about 0.1 mg/day to about 25 g/day; about 0.1 mg/day to about 10 g/day; about 0.1 mg to about 3 g/day; or about 0.1 mg to about 1 g/day, in single, divided, or continuous doses (which dose may be adjusted for the patient's weight in kg, body surface area in m2, and age in years). An effective amount of a pharmaceutical agent is that which provides an objectively identifiable improvement as noted by the clinician or other qualified observer. As used herein, the term “dosage effective manner” refers to amount of an active compound to produce the desired biological effect in a subject or cell.

Many pharmaceutical compositions are administered as a fixed dose, at regular intervals, to achieve therapeutic efficacy. The duration of action is reflected by the active ingredient's (e.g., a compound encompassed by Formula (I), (II), (III), or (IV)) plasma half-life. Since efficacy is often dependent on sufficient exposure within the central nervous system administration of CNS drugs with a short half-life may require frequent maintenance dosing. Advantageously disclosed herein are methods of treating, preventing, or mitigating seizures and seizure disorders by administration of a pharmaceutical composition including a compound encompassed by Formula (I), (II), (III), or (IV) or a pharmaceutically acceptable salt thereof. For example, in embodiments, methods of treating an seizure or seizure disorder are provided which include administering to a patient in need thereof a pharmaceutical composition including about 0.05 mg to about 2000 mg of a compound encompassed by Formula (I), (II), (III), or (IV), wherein the composition provides improvement for more than 6 hours after administration to the patient. For example, in embodiments, methods of treating an seizure or seizure disorder are provided, which include administering to a patient in need thereof a pharmaceutical composition including about 0.05 mg to about 75 mg of a compound encompassed by Formula (I), (II), (III), or (IV) or pharmaceutically acceptable salt thereof, wherein the composition provides improvement for more than 6 hours after administration to the patient.

In embodiments, methods of treating a seizure or a seizure disorder include administering to a patient in need thereof a pharmaceutical composition including about 0.05 mg to about 50 mg of a compound encompassed by Formula (I), (II), (III), or (IV)) or a pharmaceutically acceptable salt thereof. In embodiments, methods of treating a seizure or a seizure disorder include administering to a patient in need thereof a pharmaceutical composition including about 0.1 mg to about 30 mg of a compound encompassed by Formula (I), (II), (III), or (IV)) or a pharmaceutically acceptable salt thereof. For example, dosages may include amounts of a compound encompassed by Formula (I), (II), (III), or (IV) or pharmaceutically acceptable salt thereof in the range of about, e.g., 0.05 mg to 50 mg, 1 mg to 30 mg, 1 mg to 20 mg, 1 mg to 15 mg, 0.01 mg to 10 mg, 0.1 mg to 15 mg, 0.1 mg to 30 mg, 0.15 mg to 12.5 mg, or 0.2 mg to 10 mg, with doses of 0.05 mg, 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12 mg, 15 mg, 20 mg, 25 mg, and 30 mg being specific examples of doses.

Typically, dosages of a compound encompassed by Formula (I), (II), (III), or (IV) or a pharmaceutically acceptable salt thereof are administered once or twice daily to a patient in need thereof. The methods and compositions described herein may provide reduced dosing frequency and reduced adverse events and/or increased efficacy. In embodiments, the dosage is about, e.g., 0.05-30 mg/day, 0.1-20 mg/day, or 0.2-15 mg/day, or 0.5-10 mg/day, or 0.75-5 mg/day, for example 0.1 mg/day, 0.2 mg/day, 0.5 mg/day, 0.75 mg/day, 1 mg/day, 1.5 mg/day, 2 mg/day, 3 mg/day, 4 mg/day, 5 mg/day, 6 mg/day, 7 mg/day, 8 mg/day, 9 mg/day, 10 mg/day, 11 mg/day, 12 mg/day, 13 mg/day, 14 mg/day, 15 mg/day, 16 mg/day, 17 mg/day, 18 mg/day, 19 mg/day, 20 mg/day, 21 mg/day, 22 mg/day, 23 mg/day, 24 mg/day, 25 mg/day, 26 mg/day, 27 mg/day, 28 mg/day, 29 mg/day, or 30 mg/day.

In embodiments, a compound encompassed by Formula (I), (II), (III), or (IV) or a pharmaceutically acceptable salt thereof is administered at doses of 0.2 mg to 1 mg in infants or 1-20 mg in adults once daily.

In embodiments, the pharmaceutical compositions include 0.1 mg to 25 mg, 0.1 mg to 20 mg, 0.1 mg to 15 mg, 0.5 mg to 25 mg, 0.5 mg to 20 mg, 0.5 to 15 mg, 1 mg to 25 mg, 1 mg to 20 mg, 1 mg to 15 mg, 1.5 mg to 25 mg, 1.5 mg to 20 mg, 1.5 mg to 15 mg, 2 mg to 25 mg, 2 mg to 20 mg, 2 mg to 15 mg, 2.5 mg to 25 mg, 2.5 mg to 20 mg, 2.5 mg to 15 mg, 3 mg to 25 mg, 3 mg to 20 mg, 3 mg to 15 mg of a compound encompassed by Formula (I), (II), (III), or (IV)) or a pharmaceutically acceptable salt thereof.

In embodiments, the pharmaceutical compositions include 5 mg to 20 mg, 5 mg to 10 mg, 4 mg to 6 mg, 6 mg to 8 mg, 8 mg to 10 mg, 10 mg to 12 mg, 12 mg to 14 mg, 14 mg to 16 mg, 16 mg to 18 mg, or 18 mg to 20 mg of a compound encompassed by Formula (I), (II), (III), or (IV)) or a pharmaceutically acceptable salt thereof.

In embodiments, the pharmaceutical compositions include 0.1 mg, 0.25 mg, 0.5 mg, 1 mg, 2.5 mg, 3 mg, 4 mg, 5 mg, 7 mg, 7.5 mg, 10 mg, 12.5 mg, 15 mg, 17.5 mg, 20 mg, 22.5 mg, 25 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 225 mg, 250 mg, 300 mg, 350 mg, 400 mg, 500 mg, 600 mg, 700 mg, 800 mg, 900 mg, or 1000 mg of a compound encompassed by Formula (I), (II), (III), or (IV) or a pharmaceutically acceptable salt thereof or amounts that are multiples of such doses. In embodiments, the pharmaceutical compositions include 2.5 mg, 5 mg, 7.5 mg, 10 mg, 15 mg, or 20 mg of a compound encompassed by Formula (I), (II), (III), or (IV)) or a pharmaceutically acceptable salt thereof.

In embodiments, the total amount of a compound encompassed by Formula (I), (II), (III), or (IV) or pharmaceutically acceptable salt thereof administered to a subject in a 24-hour period is 1 mg to 100 mg. In embodiments, the total amount of a compound encompassed by Formula (I), (II), (III), or (IV) or pharmaceutically acceptable salt thereof administered to a subject in a 24-hour period is 1 mg to 50 mg. In embodiments, the total amount of a compound encompassed by Formula (I), (II), (III), or (IV) or pharmaceutically acceptable salt thereof administered to a subject in a 24-hour period is 1 mg to 20 mg. In embodiments, the total amount of a compound encompassed by Formula (I), (II), (III), or (IV) or pharmaceutically acceptable salt thereof administered to a subject in a 24-hour period is 5 mg, 10 mg, 15 mg or 20 mg. In embodiments, the subject may be started at a low dose and the dosage is escalated. In this manner, it can be determined if the drug is well tolerated in the subject. Dosages can be lower for children than for adults. In embodiments, a dose of a compound encompassed by Formula (I), (II), (III), or (IV) for children can be 0.1 mg/kg to 1 mg/kg.

In embodiments, a method of treating a seizure or seizure disorder includes administering a compound encompassed by Formula (I), (II), (III), or (IV) or a pharmaceutically acceptable salt thereof to a patient in need thereof. A compound encompassed by Formula (I), (II), (III), or (IV) or a pharmaceutically acceptable salt thereof can be administered in doses ranging from 10 mg/kg to 40 mg/kg, e.g., 11 mg/kg to 39 mg/kg, 12 mg/kg to 38 mg/kg, 13 mg/kg to 37 mg/kg, 14 mg/kg to 36 mg/kg, 15 mg/kg to 35 mg/kg, 16 mg/kg to 34 mg/kg, 17 mg/kg to 33 mg/kg, 18 mg/kg to 32 mg/kg, 19 mg/kg to 31 mg/kg, 20 mg/kg to 30 mg/kg, 21 mg/kg to 29 mg/kg, 22 mg/kg to 28 mg/kg, 23 mg/kg to 27 mg/kg, or 24 mg/kg to 26 mg/kg. In embodiments, a compound encompassed by Formula (I), (II), (III), or (IV) or a pharmaceutically acceptable salt thereof can be administered at a dose of, for example, 1 mg, 10 mg, 20 mg, 30 mg, 40 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1225 mg, 1250 mg, 1275 mg, 1300 mg, 1325 mg, 1350 mg, 1375 mg, 1400 mg, 1425 mg, 1450 mg, 1475 mg, 1500 mg, 1525 mg, 1550 mg, 1575 mg, 1600 mg, 1625 mg, 1650 mg, 1675 mg, 1700 mg, 1725 mg, 1750 mg, 1775 mg, 1800 mg, 1825 mg, 1850 mg, 1875 mg, 1900 mg, 1925 mg, 1950 mg, 1975 mg, or 2000 mg.

The pharmaceutical compositions can be included in a container, pack, or dispenser together with instructions for administration.

The composition of the invention is capable of further forming salts. The composition of the invention is capable of forming more than one salt per molecule, e.g., mono-, di-, tri-. All of these forms are also contemplated within the scope of the claimed invention.

Exemplary pharmaceutically acceptable salts refer to derivatives of the compounds encompassed by Formula (I), (II), (III), or (IV), wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present invention also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

It should be understood that all references to pharmaceutically acceptable salts include solvent addition forms (solvates) or crystal forms (polymorphs) as defined herein, of the same salt.

The composition of the invention may also be prepared as esters, for example, pharmaceutically acceptable esters. For example, a carboxylic acid function group in a compounds of formula (I), (II), (III), or (IV) or a pharmaceutically acceptable salt thereof can be converted to its corresponding ester, e.g., a methyl, ethyl or other ester. Also, an alcohol group in a compound can be converted to its corresponding ester, e.g., acetate, propionate or other ester.

The composition of the present invention can also be prepared as prodrugs, for example, pharmaceutically acceptable prodrugs. The terms “pro-drug” and “prodrug” are used interchangeably herein and refer to any compound which releases an active parent drug in vivo. Since prodrugs are known to enhance numerous desirable qualities of pharmaceuticals (e.g., solubility, bioavailability, manufacturing, etc.), the compounds of the present invention can be delivered in prodrug form. Thus, the present invention is intended to cover prodrugs of the presently claimed compounds, methods of delivering the same and compositions containing the same. “Prodrugs” are intended to include any covalently bonded carriers that release an active parent drug of the present invention in vivo when such prodrug is administered to a subject. Prodrugs in the present invention are prepared by modifying functional groups present in the compound in such a way that the modifications are cleaved, either in routine manipulation or in vivo, to the parent compound. Prodrugs include compounds of the present invention wherein a hydroxy, amino, sulfhydryl, carboxy or carbonyl group is bonded to any group that may be cleaved in vivo to form a free hydroxyl, free amino, free sulfhydryl, free carboxy or free carbonyl group, respectively.

Examples of prodrugs include, but are not limited to, esters (e.g., acetate, dialkylaminoacetates, formates, phosphates, sulfates and benzoate derivatives) and carbamates (e.g., N,N-dimethylaminocarbonyl) of hydroxy functional groups, esters (e.g., ethyl esters, morpholinoethanol esters) of carboxyl functional groups, N-acyl derivatives (e.g., N-acetyl)N-Mannich bases, Schiff bases and enaminones of amino functional groups, oximes, acetals, ketals and enol esters of ketone and aldehyde functional groups in compounds of the invention, and the like, See Bundegaard, H., Design of Prodrugs, p1-92, Elesevier, New York-Oxford (1985).

The composition, or pharmaceutically acceptable salts, esters or prodrugs thereof, are administered orally, nasally, transdermally, pulmonary, inhalationally, buccally, sublingually, intraperintoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the invention can be found in Remington: the Science and Practice of Pharmacy, 19.sup.th edition, Mack Publishing Co., Easton, Pa. (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

In various embodiments, compositions can include diluents of various buffer content (e.g., Tris —HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., Thimersol, benzyl alcohol) and bulking substances (e.g., lactose, mannitol); incorporation of the material into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. See, e.g., Remington's Pharmaceutical Sciences, 18th Ed. (1990, Mack Publishing Co., Easton, Pa. 18042) pages 1435-1712 which are herein incorporated by reference. A pharmaceutical composition of the present invention can be prepared, for example, in liquid form, or can be in dried powder, such as lyophilized form. Particular methods of administering such compositions are described infra.

### Methods For Treating And Preventing Seizures And Seziure Disorders

The compounds and compositions disclosed herein can be used for treating, preventing, or mitigating seizures and various conditions associated with seizures. Methods are provided for treatment, prevention, and mitigation of seizures associated with epileptic disorders including epilepsy, epilepsy with generalized tonic-clonic seizures, epilepsy with myoclonic absences, frontal lobe epilepsy, temporal lobe epilepsy, Landau-Kleffner Syndrome, Ohtahara syndrome, Rasmussen's syndrome, West's syndrome, Lennox-Gastaut syndrome (LGS), Rett syndrome, CDKL5 disorder, childhood absence epilepsy, essential tremor, Dravet syndrome, Doose syndrome, acute repetitive seizures, benign rolandic epilepsy, status epilepticus, refractory status, epilepticus, super-refractory status epilepticus (SRSE), PCDH19 pediatric epilepsy, increased seizure activity or breakthrough seizures (increased seizure activity, also called serial or cluster seizures and sodium channel protein type 1 subunit alpha (Scnla)-related disorders by administering to a patient in need thereof a pharmaceutical composition containing a compound of formula (I), (II), (III), or (IV) or a pharmaceutically acceptable salt thereof.

In embodiments, methods are provided for treatment, prevention, or mitigation of seizures and seizures associated with epileptic disorders including status epilepticus, benign rolandic epilepsy (BRE), intractable childhood epilepsy (ICE), childhood absence epilepsy (CAE), juvenile myoclonic epilepsy (JME), infantile spasms (or West syndrome), Dravet syndrome and Lennox-Gastaut syndrome (LGS) by administering to a patient in need thereof a pharmaceutical composition including a compound of formula (I), (II), (III), or (IV), or a pharmaceutically acceptable salt thereof.

In embodiments, methods are provided for treatment, prevention, or mitigation of seizures and seizures related to epileptic disorders characterized as a sodium channel protein type 1 subunit alpha (Scnla)-related disorder. Sonla-related disorders include generalized epilepsy with febrile seizures plus, intractable childhood epilepsy with generalized tonic-clonic seizures, intractable infantile partial seizures, myoclonic-astatic epilepsy, severe myoclonic epilepsy in infancy, simple febrile seizures, Dravet syndrome, Lennox-Gastaut syndrome (LGS), infantile spasms, and vaccine-related encephalopathy and seizures.

In embodiments, methods are provided for treatment of a sodium channel protein type 1 subunit alpha (Scnla)-related disorder by administering to a patient in need thereof a pharmaceutical composition including a compound of formula (I), (II), (III), or (IV), or a pharmaceutically acceptable salt thereof.

The methods described herein may also be effective in subjects experiencing intractable seizures, status epilepticus, akinetic seizures, myoclonic seizures, absence seizures, or severe myoclonic epilepsy in infancy (SMEI). In embodiments, the disorders are characterized by intractable seizures. Intractable seizures (also referred to as “uncontrolled” or “refractory” seizures) are seizures that cannot be controlled with conventional treatments. For example, the subject can have intractable epilepsy or another disorder characterized by intractable seizures, or a disorder characterized by status epilepticus. Status epilepticus is a condition in which seizures follow one another without recovery of consciousness between them. Accordingly, in embodiments, the disclosed methods are used to treat subjects that would otherwise be resistant to one or more conventional therapies.

The methods described herein may be particularly useful for treating children and infants, and for treating disorders that onset during infancy or childhood. In embodiments, the subject of the disclosed method is a newborn, a baby, a toddler, a preschooler, a school-age child, a tween, or a teenager. In embodiments, the subject is 18 years old or younger, 12 years old or younger, 10 years old or younger, 8 years old or younger, 6 years old or younger, 4 years old or younger, 2 years old or younger, 1 year old or younger. In embodiments, the subject is an adult that is over eighteen years old.

In embodiments, the epileptic disorders are characterized by seizures associated with epilepsy. In embodiments, the seizures are non-epileptic seizures (NES) or dissociative seizures that are distinguished from epilepsy. Non-epileptic seizures include organic non-epileptic seizures and psychogenic seizures.

Epilepsy is a neurological disorder that occurs when nerve cell activity in the brain becomes disrupted, leading to seizures or periods of unusual behavior, sensations and sometimes loss of consciousness. A subject can be said to have epilepsy when having two seizures without an obvious cause. Epilepsy can occur in both adults and children, and can be associated with a specific syndrome. Accordingly, in embodiments, the subject has a childhood epilepsy syndrome such as benign rolandic epilepsy (BRE), childhood absence epilepsy (CAE), juvenile myoclonic epilepsy (JME), infantile spasms (or West syndrome), Dravet syndrome or Lennox-Gastaut syndrome (LGS).

In embodiments, the subject does not experience diagnosable seizures, but exhibits subclinical electrical discharges, which refers to a high rate of seizure-like activity when their brain waves are measured with an electroencephalogram. Epileptic syndromes associated with these seizure-like discharges include Landau-Kleffner Syndrome, Dravet syndrome and Continuous Spike-wave Activity during Slow-wave Sleep.

In embodiments, the epileptic disorders treated by the methods and compositions described herein include Scn1A-related seizure disorders. Scn1A-related seizure disorders include simple febrile seizures (FS) and generalized epilepsy with febrile seizures plus (GEFS+) at the mild end to Dravet syndrome and intractable childhood epilepsy with generalized tonic-clonic seizures (ICE-GTC) at the severe end. Specific Scn1A-related seizure disorders include, but are not limited to, generalized epilepsy with febrile seizures, intractable childhood epilepsy with generalized tonic-clonic seizures, intractable infantile partial seizures, myoclonic-astatic epilepsy, severe myoclonic epilepsy in infancy, simple febrile seizures, Dravet syndrome, Lennox-Gastaut syndrome (LGS), infantile spasms, and vaccine-related encephalopathy.

In embodiments, the subject has an intellectual epileptic disability (IDD) such as an Autism Spectrum Disorders (ASD). In embodiments, the subject of the disclosed method has epilepsy and an IDD or ASD disorder. Common IDD and ASD that are comorbid with seizures and epilepsy include, but are not limited to, fragile X syndrome (FXS), Rett syndrome (RTT), Angelman syndrome, Prader-Willi syndrome, Velocardiofacial syndrome, Smith-Lemli-Opitz syndrome, neuroligin mutations and “interneuronopathies” resulting from aristaless-related homeobox, X-linked (ARX) and Nueropilin 2 (NRP2) gene mutations.

Also provided herein are methods and compositions for treating seizures and seizure disorders by co-administering to a patient in need thereof composition comprising a compound encompassed by Formula (I), (II), (III), or (IV), or a pharmaceutically acceptable salt thereof, and an anti-epileptic drug, for example, sodium valproate, carbamazepine, lamotrigine, levetiracetam, or topiramate.

The compounds of Formula (I), (II), (III), or (IV) or pharmaceutically acceptable salts thereof and compositions thereof can be used to treat, prevent, mitigate seizures and seizure disorders. A seizure is a burst of uncontrolled electrical activity between brain cells (also called neurons or nerve cells) that causes temporary abnormalities in muscle tone or movements (stiffness, twitching or limpness), behaviors, sensations or states of awareness. Seizures are not all alike. A seizure can be a single event due to an acute cause, such as medication. When a person has recurring seizures, this is known as epilepsy. Seizures symptoms vary and can include a sudden change in awareness or full loss of consciousness, unusual sensations or thoughts, involuntary twitching or stiffness in the body or severe stiffening and limb shaking with loss of consciousness (a convulsion.)

Seizure disorders are disorders that can directly or indirectly cause a seizure and include two major classes or groups of seizures: focal onset and generalized onset. Focal onset seizures start in one area and can spread across the brain and cause mild or severe symptoms, depending on how the electrical discharges spread. Generalized seizures can start as focal seizures that spread to both sides of the brain. They also can occur as “generalized onset” seizures in which seizure activity starts simultaneously over both sides of the brain. Generalized onset seizures usually start during childhood and are similar to a thermostat surge or a light flash-abnormal regulation between parts of the brain causes the seizures.

Focal seizures are also called partial seizures since they begin in one area of the brain. They can be caused by any type of focal injury that leaves scar tangles. Medical history or MRI will identify a cause (such as trauma, stroke or meningitis) in about half of the people who have focal seizures. Developmental scars—ones that occur as part of fetal and early growth of the brain—are common causes of focal seizures in children.

Focal seizures can start in one part of the brain and spread to other areas, causing symptoms that are mild or severe, depending on how much of the brain becomes involved.

At first, the person may notice minor symptoms, which is referred to as an aura. The person may have altered feelings or sense that something is about to happen (premonition). Some people experiencing an aura describe a rising sensation in the stomach similar to riding on a roller coaster. As the seizure spreads across the brain, more symptoms appear. If the abnormal electrical activity involves a large area of the brain, the person may feel confused or dazed, or experience minor shaking, muscle stiffening, or fumbling or chewing motions. Focal seizures that cause altered awareness are called focal unaware seizures or complex partial seizures. The electrical activity of the seizure can remain in one sensory or motor area of the brain, resulting in a focal aware seizure (also called simple partial seizure). The person is aware of what is happening, and may notice unusual sensations and movements. Focal seizures can evolve into major events that spread to the entire brain and cause tonic-clonic seizures. These seizures are important to treat and prevent since they can cause respiratory problems and injuries.

Generalized-onset seizures are surges of abnormal nerve discharges throughout the cortex of the brain more or less at the same time. The most common cause is an imbalance in the “brakes” (inhibitory circuits) and “accelerator” (excitatory circuits) of electrical activity in the brain. Generalized seizures may have a genetic component, but only a small number of people with generalized seizures have family members with the same condition. There is a slight increase in risk for generalized seizures in the children or other family members of an affected person with generalized seizures, but the severity of the seizures can vary from person to person. Genetic testing may reveal a cause for generalized seizures. Sleep deprivation or drinking large amounts of alcohol can increase the excitatory response and increase the risk of generalized-onset seizures, especially in people with a genetic tendency to have them.

Childhood absence epilepsy shows up as brief staring episodes in children, usually starting between ages four and six. Children usually outgrow these. Juvenile absence epilepsy starts slightly later and can persist into adulthood; people with these kinds of seizures may develop tonic-clonic seizures in addition to absence of seizures in adulthood.

Myoclonic seizures consist of sudden body or limb jerks that can involve the arms, head and neck. The spasms occur on both sides of the body in clusters, especially in the morning. When these seizures develop in adolescence along with tonic-clonic seizures, they are part of a syndrome called juvenile myoclonic epilepsy. People can also have myoclonic seizures as part of other epilepsy related-conditions.

Some people, usually those with multiple brain injuries and intellectual disability, have tonic seizures consisting of sudden stiffness in the arms and body, which can cause falls and injuries. Many persons with tonic seizures have a syndrome called Lennox Gastaut syndrome. This condition may involve intellectual disability, multiple seizure types including tonic seizures. People with Lennox Gastaut syndrome can have a distinctive EEG pattern called slow spike and wave.

Persons with diffuse brain disorders also may have atonic seizures, characterized by a sudden loss of body tone that results in collapsing, often with injuries. A sequence of a brief tonic episode followed by an atonic seizure is called a tonic-atonic seizure. Tonic and atonic seizures are often managed with specific medications and sometimes with nerve stimulation and diet therapies.

Tonic-clonic seizures can evolve from any of the focal or generalized seizure types. For example, a focal seizure can spread to both sides of the brain and cause tonic-clonic seizures. A cluster of myoclonic seizures can become continuous and evolve into a tonic-clonic seizure. Generalized onset tonic-clonic seizures can occur alone or as part of another syndrome such as juvenile myoclonic epilepsy (JME) or juvenile absence epilepsy during adulthood.

The following examples are provided to illustrate certain embodiments of the invention. They are not intended to limit the invention in anyway. Such modifications are intended to fall within the scope of the appended claims.

### EXAMPLES

### Example 1

### Synthesis

Utilizing a simple bioisosteric replacement of the 5th position methyl group hydrogen atoms with fluorine atoms, results in increased electronegativity. Optimization of the lipophilic character for the enaminone intermediate building blocks ultimately lead to improvement of the overall drug-likeness of the target analog (7). (Scheme 1)

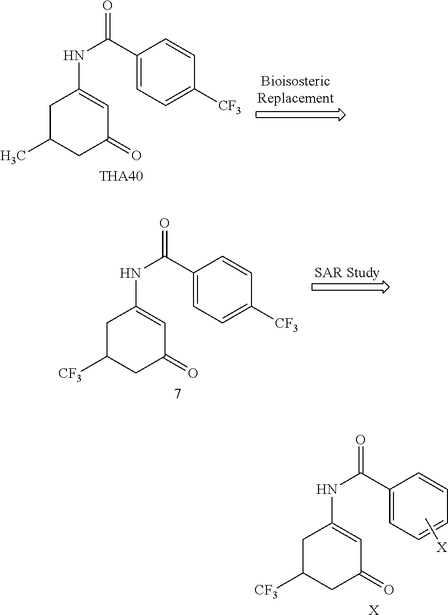

Utilizing SimulationPlus® physicochemical properties prediction tools MedChem Designer and ADMETPredictor 10.0 during drug design has become an effective strategy used by medicinal chemists, with the most traditional predictor being the Lipinski's rule of five. The predicted values are typically indicators of absorption, distribution, and bioactivity of a potential drug molecule. Optimal LogP values for CNS drugs, have a threshold of ˜3 and the TPSA value for CNS drugs with optimal penetration is less than 70 Å. New compound (7), has values that are within these ranges. The predicted values generated from the physicochemical properties' prediction tool, further validates our lead optimization strategy as being most advantageous for the development of potential anticonvulsants.

The synthesis of diketones involves the condensation of keto-esters and Michael acceptors, followed by ester hydrolysis and decarboxylation under acidic conditions. 31 This was done using a tert-butyl keto-ester (8c) rather than the methyl and ethyl keto-ester with the same Michael acceptor ethyl 4, 4, 4-trifluorocrotonate (9). The Michael addition step is followed by intramolecular aldo condensation in the presence of the base. This intramolecular cyclization of the enolate anion proceeds via a Claisen condensation and the use of 8c prevents a reverse Claisen reaction during the hydrolysis and decarboxylation step. The reaction was refluxed in a solution of sodium tertbutoxide resulting in a high yield of the inorganic sodium enolate of 5-(trifluoromethyl) cyclohexane-1,3-dione (10) as seen in Scheme 2.

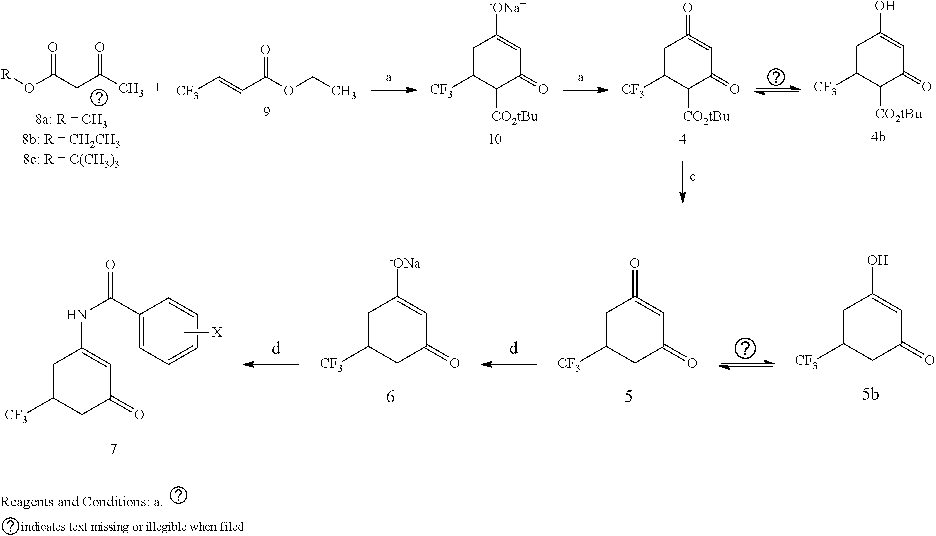

The sodium enolate was acidified using concentrated hydrochloric acid to liberate the ester intermediate (4) which underwent an acid catalyzed hydrolysis and decarboxylation by refluxing in 0.05N H₂SO₄ for two hours to provide the fluorinated diketone (5) in a high yield. (Scheme 2). The resulting diketone then underwent an amination reaction to yield the trifluoromethylated enaminone intermediate (6) in a significantly high yield (Scheme 2). Upon successful synthesis of the trifluoromethylated enaminone, an acylation reaction generating the bioisosteric analog of lead compound THA40. The reaction proceeded in an N-acylation method with the first step being the generation of the enaminone anion by refluxing in a mixture of anhydrous THF with sodium hydride (NaH) as base followed by an N-acylation at room temperature.

### Results and Discussion

The use of tert-butyl acetoacetate rather than ethyl or methyl acetoacetate as a choice of the Michael adduct in the condensation reaction was established by Friary's research group as a means to overcome reverse Claisen condensation reaction. Previous reports highlight the formation of an unwanted ester byproduct when methoxide or ethoxide is used and absent when tert-butoxide, a stronger base is used as an alternative since the base is a poor neuclophile. The poor nucleophilicity of the tert-butyl group on both the base of choice (sodium tert-butoxide) and Michael donor (8c) facilitated the rapid formation of the sodium enolate precipitate in 80% yield after two hours of reflux. We propose that the reason for this is due to the insusceptibility of the tert-butyl diketo-ester (4) to undergo nucleophilic attack by the alkoxide base, resulting in a more stable intermediate.

We focused on extracting the organic ester (4) from the inorganic sodium enolate by simply dissolving the inorganic salt in water and acidified the aqueous mixture using concentrated hydrochloric acid, which resulted in the formation of the ester as a white precipitate that was extracted in 73% yield. Next, the tert-butyl ester group is removed by a simple hydrolysis, followed by a decarboxylation in dilute aqueous sulfuric acid. We were able to reproduce this method and achieve (5) as white crystals in 75% yield. The integrity of the cyclic product was preserved as confirmed by NMR and GCMS analytical methods. To investigate the novelty of our synthetic strategy, a SciFinder search for the synthesis of 4-carbo-tert-butoxy-5-trifluoromethylcyclohexane-1,3-dione (4) and N-(3-oxo-5-(trifluoromethyl) cyclohex-1-en-1-yl)-4-(trifluoromethyl)benzamide (7) was conducted. The results for compound (7) yielded no synthetic methods. However, the search for compound (4) showed one previous synthetic method with a different reaction condition and low yield reported by Jackson et al. Therefore, to date no other research group has attempted the method reported in this paper for liberation of the fluorinated diketo ester. This ester intermediate has other applications such as, its use as a building block for synthesis of other potential therapeutic agents, where an ester intermediate is required. In this work, we were able to utilize the ester intermediate as a building block for improved and efficient synthesis of the diketone intermediate (5).

The conversion of 1, 3-diketones to enaminones in significant yield has been established by Baraldi et. al. 9-12, 14, 32-35 We successfully converted compound 5 to 6 via a reflux of compound 5 in benzene, using ammonium acetate as source of amine and a Dean-Stark trap for the azeotropic removal of water. The enaminone 6 was formed in 95% yield after recrystallization in benzene/MeOH.

A comparison of the reaction conditions for the derivation of the fluorinated intermediates is shown in Table 1.

TABLE 1
Table of Drug-likeness

| | Molecular | | | | | | | | |
|---|---|---|---|---|---|---|---|---|---|
| Compound | Formula | MW^{a} | mLogP^{b} | nROTB^{c} | nHBA^{d} | nHBD^{e} | TPSA^{f} | nViol^{g} | MR^{h} |
| THA40 | C₁₅H₁₄F₃NO₂ | 297.27 | 2.71 | 4 | 5 | 1 | 46.17 | 0 | 70.89 |
| GSA62 | C₁₅H₁₃F₄NO₂ | 315.26 | 3.1 | 4 | 6 | 1 | 46.17 | 0 | 70.85 |
| TTA35 | C₁₅H₁₄F₃NO₃ | 313.27 | 1.86 | 5 | 6 | 1 | 55.4 | 0 | 72.58 |
| IAA61 | C₁₅H₁₄F₃NO₂ | 297.27 | 2.71 | 4 | 5 | 1 | 46.17 | 0 | 70.89 |
| IAA52 | C₁₅H₁₄F₃NO₃ | 313.27 | 1.86 | 5 | 6 | 1 | 55.4 | 0 | 72.58 |
| 7 | C₁₅H₁₁F₆NO₂ | 351.24 | 3.07 | 5 | 8 | 1 | 46.17 | 0 | 71.09 |

^{a}Molecular weight (<500 g/mol); ^{b}Morugichi LogP (<4.15); ^{c}Number of rotatable bonds (<10); ^{d}Number of hydrogen bond acceptors (O, N & F atoms <10); ^{e}Number of hydrogen bond donors (OH and NH groups <5); ^{f}Topological surface area (<70); ^{g}Number of violations; ^{h}Molar refractivity between 40-130.

Aside from the formation of unwanted by products in the reaction, keto-esters (8a) & (8b) and their respective alkoxide bases produced relatively low yields, despite the long reaction times as outlined in Table 1. Switching to a less reactive and stronger base tert-butoxide, increased the yield regardless of the starting keto-ester and the length of the reaction time as shown in the Table 1. With the use of (8c) as the starting of keto-ester, we were able to produce a higher yield of the sodium enolate (10) and successfully synthesize a novel diketo-ester.

TABLE 2
Comparative Synthesis of Fluorinated
Cyclohexanedione intermediates

| | | | Reaction | Yield (%) | | | |
|---|---|---|---|---|---|---|---|
| Keto-ester | Base (equiv) | Solvent | time (h) | 10 | 4 | 5 | 6 |
| 8a | MeONa (1.0 | MeOH | 6-8 | — | — | 20¹¹ | — |
| | equiv) | | | | | | |
| 8a | t-BuONa | ^{t}-BuOH | 6-8 | — | — | 45¹¹ | 89¹¹ |
| | (1.0 equiv) | | | | | | |
| 8a | t-BuONa | ^{t}-BuOH | 2 | 46 | — | 98¹² | — |
| | (1.0 equiv) | | | | | | |
| 8b | EtONa (1.0 | EtOH | 6-8 | — | — | 26¹¹ | — |
| | equiv) | | | | | | |
| 8c | t-BuONa | ^{t}-BuOH | 2 | 80 | 73 | 75 | 95 |
| | (1.0 equiv) | | | | | | |

Enaminone (6) was successfully converted to the target analog (7) with the use of 3 molar equivalences of the sodium hydride base. One of the chemical properties of the enaminone system is the “push-pull system” of the vinylogous amide, thus resulting in poor nucleophilicity of the primary amine. As a result, a strong base such as NaH was used for the N-deprotonation step of the reaction. After N-deprotonation, the N-acylation was done by adding the 4-trifluoromethyl benzoyl chloride in a dropwise fashion and the reaction was monitored via TLC and GCMS, until there was indication of reaction completion, followed by work up to extract the final product in good yield of 54.6%.

From the new drug series, target analog (7) was evaluated by ETSP in the preliminary identification anticonvulsant screening test. Compared to the lead compound THA40, the CF3 bioisosteric analog (7) was not as efficacious against seizure protection in the acute seizure rodent models. Whereas THA40 was able to elicit a pharmacological response and suppress seizures at a moderate dose of 150 mg/kg in the 6 Hz 44 mA animal model as previously mentioned. This new analog produced pharmacological response at a higher dose of 300 mg/kg and only 25% protection was observed, as compared to lead compound THA40. Interestingly, while THA40 did not have any activity in the maximal electroshock (MES) model which is the gold standard for anticonvulsant activity screening, compound 7 showed moderate activity at 100 mg/kg with 25% after 2 h. At a higher dose of 300 mg/kg, 25% of the animals were protected both at 0.5 h and 2 h.

### Chemistry

All chemicals and reagents were purchased from Sigma Aldrich, USA and used without further purification. NMR spectra were recorded at room temperature on a Bruker Advance series instrument (400 MHZ). GC-MS analysis was performed on Shimadzu QP-2010SE instrument. Melting point analysis was done on a ThermoFisher IA9000 series digital melting point apparatus. Elemental analysis was outsourced to Micro-Analysis Inc. located in Wilmington DE.

### Synthesis of Sodium 4-carbo-tert-butoxy-5-trifluoromethylcyclohexane-1,3-dione (10)

To a solution of freshly prepared sodium tert-butoxide, (prepared from anhydrous tertbutanol and sodium metal (67 mmol) in mineral oil), was added tert-butyl acetoacetate (67 mmol) dropwise at 0° C. The reaction mixture was stirred at 0° C. for 15 min. A solution of ethyl 4, 4, 4-trifluorocrotonate (67 mmol) in tert-butanol was added dropwise to the reaction mixture. The mixture was brought to room temperature and stirred for an additional 30 min. The reaction mixture was refluxed for 2 h where the formation of a solid white precipitate is observed. The product was collected by vacuum filtration and washed with anhydrous hexane to afford 16.5 g (80%) of (10) as a white powder left to dry for the next step which was acidification of the salt.

### Synthesis of 4-carbo-tert-butoxy-5-trifluoromethylcyclohexane-1,3-dione (4)

A solution of the inorganic salt (16 g) was dissolved in 87 ml of water and was filtered to remove impurities and acidified with 8.7 ml of hydrochloric acid. The resulting white precipitate was washed with water and air dried to give 73% of 4-carbo-tert-butoxy-5-trifluoromethyl-cyclohexane-1,3-dione (9): MP. 135-136.2° C. (lit. 133-137oC12); 1H NMR (400 MHZ, DMSO): δ, ppm 11.80 (1H, s), 5.32 (1H, s), 3.43 (2H, d, J=25.7 Hz), 2.61 (2H, d, J=17.1 Hz), 1.38 (9H, s).; Anal. Calcd for C12H15F3O4: C, 51.43; H, 5.40; F, 20.34 Found: C, 51.48; H, 5.33; F, 20.26. The sample was left to dry for the next step which is hydrolysis and decarboxylation of the ester to yield the target compound.

### Synthesis of 5-(trifluoromethyl) cyclohexane-1,3-dione (5)

To 300 ml of stirring and boiling 0.05N sulfuric acid was added, 15 g of the tert-butyl ester in small quantities over a period of 40 mins. Slow addition was necessary to avoid material loss due to excessive formation of CO₂ gas. The mixture was refluxed for 60 mins and allowed to cool down to room temperature. After cooling to room temperature, the reaction mixture was stored at 4° C. overnight to afford white crystals collected by vacuum filtration. The crude sample was recrystallized from ethyl acetate to give 75% of long white needle-like crystals of 5-trifluoromethylcyclohexane-1,3-dione, MP. 140.4-142.20C (lit. 140-1420C10); 1H NMR (400 MHZ, DMSO): δ, ppm 11.49-11.45 (1H, m), 5.27 (1H, s), 3.22-3.09 (1H, m), 2.43-2.38 (4H, m); Anal. Calcd for C7H₇F30: C, 46.68; H, 3.92; F, 31.64 Found: C, 46.62; H, 3.87; F, 31.52

### Synthesis of 3-amino-5-(trifluoromethyl) cyclohex-2-en-1-one (6)

To a 250 ml two-neck round bottom flask fitted with a condenser, Dean-Stark trap, and magnetic stirrer was added, 100 ml of anhydrous benzene under nitrogen. The reaction flask was cooled to 0° C. before the addition of 5-trifluoromethylcyclohexane-1, 3-dione (4) (12 g, 67 mmol) and ammonium acetate (10.329 g, 134 mmol). After stirring for 10 min, 5 ml of acetic acid was added dropwise and the reaction mixture was brought to room temperature and allowed to stir for 30 min. The mixture refluxed for 1 h and once cooled, was allowed to stir overnight at room temperature. The crude product precipitated as a pale yellow solid and was collected via vacuum filtration and allowed to air dry. Once dried, the crude product was recrystallized benzene/MeOH to afford the titular compound in 95%, MP. 168.5-169.60C (lit. 160-1620C10). 1H NMR (400 MHZ, DMSO): δ, ppm 6.97 (2H, d, J=89.8 Hz), 4.98 (1H, s), 3.02 (1H, s), 2.47-2.38 (2H, m), 2.16 (1H, d, J=10.2 Hz), 1.87 (1H, s); Anal. Calcd for C7H₈F3NO: C, 46.93; H, 4.50; N, 7.82; F, 31.82 Found: C, 45.78; H, 4.70; N, 7.74; F, 31.82

### Synthesis of N-(3-oxo-5-(trifluoromethyl) cyclohex-1-en-1-yl)-4 (trifluoro-methyl)-benzamide (7)

Into a 250 ml. two-neck round bottom flask equipped with a condenser and magnetic stirrer was added 40 mL anhydrous tetrahydrofuran (THF) under nitrogen. After cooling on an ice bath, sodium hydride (402 mg, 25.2 mmol) was slowly added; followed by additional 20 mL of dry THF. 3-amino-5-(trifluoromethyl) cyclohex-2-en-1-one (1.5 g, 8.7 mmol) was added slowly over the course of 5 mins followed by 10 mL of dry THF. The reaction mixture was allowed to reflux for 40 mins. Once cooled to room temperature, the mixture was placed on an ice bath for 5 min before adding the substituted benzoyl chloride (1.24 mL, 8.7 mmol) in 15 mL of dry THF via dropping funnel. The dropping funnel was rinsed with an additional 10 mL of dry THF. The reaction mixture was stirred in an ice bath for about 10 mins. Before removing the reaction mixture from the ice bath, a first aliquot was removed and analyzed. Reaction mixture was stirred at room temperature with monitoring done by TLC and GCMS until completion. Upon confirmation of reaction completion, the reaction mixture was quenched with 80 mL deionized (DI) water and acidified with 8 mL of concentrated HCl. The aqueous solution was extracted with dichloromethane (2×75 mL) and the organic layer washed with 75 mL of 10% NaHCO₃ and 75 mL DI water. The organic layer was dried over MgSO₄, filtered, and concentrated in vacuo to yield a yellowish-white solid residue that was triturated with anhydrous ether. The product was collected via vacuum filtration to give the titular compound as white powder in 54.7% yield (1.609 g), MP 206.8-208° C. 1H-NMR (400 MHZ, DMSO) δ 10.37 (s, 1H), 8.13-8.09 (m, 2H), 7.93-7.89 (m, 2H), 6.88 (d, 1H, J=1.4 Hz), 3.00 (dd, 1H, J=4.6, 17.3 Hz), 2.81-2.72 (m, 1H). Anal. Calcd for C15H11F6NO2: C, 51.29; H, 3.16; N, 3.99; F, 32.45 Found: C, 51.15; H, 3.15; N, 3.85; F, 32.30.

### General synthesis of the N-(5-methyl-3-oxocyclohex-1-en-1-yl)-3-((trifluoromethyl)thio)benzamide

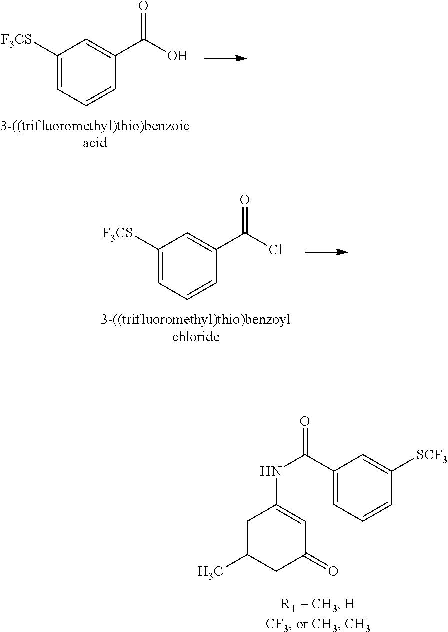

Synthetic method for 3-((trifluoromethyl)thio)benzoyl chloride: The reaction is carried under an inert N₂ atmosphere. Anhydrous toluene is used as the reaction environment. Water is expected to be generated in the reaction therefore any anhydrous byproducts is removed via azeotropic conditions. The reaction mixture will contain 3-(trifluoromethylthio)benzoic, dimethyl formamide, and thionyl chloride. The thionyl chloride is added to the reaction slowly and dropwise. The reaction is heated and refluxed for approx. 5 hours. The reaction is monitored for completion via TLC and GC-MS techniques. The acylation reaction is carried out with 3-((trifluoromethyl)thio)benzoyl chloride to yield the target N-benzamide enaminone analog using methods known in the chemical arts.

Synthesis of Pyridine Analogs of the invention. This general method may be extended to synthesized pyridine derivatives of the compounds of the invention. The starting material for synthesis is an acid chloride. A similar one-pot acylation reaction may be used to make these compounds the forgoing synthesis may be modified using known techniques.

### Pharmacology

Initial evaluation for anticonvulsant activity was done by the Epilepsy Therapy Screening Program Branch, National Institute of Neurological Disorders and Stroke. Phase I evaluation included three tests: maximal electroshock (MES), 6 Hz, and rotarod test for neurological toxicity (Tox). The studies were done in normal adult male Carworth Farms no. 1 (CF1) mice using four or more mice pre-test. Intraperitoneal (ip) administration of the test compounds was carried out as a suspension in 0.5% methylcellulose. The compounds were tested at doses ranging from 30 mg/kg to 300 mg/kg. The animals were pretreated with the test compound at time intervals of 0.5 h and 2 h. The rotarod neurologic toxicity test was completed at each dose and time point. The MES test is a high frequency (60 Hz), short duration (0.3 s) stimulus delivered through corneal electrodes in normal rodents while the 6 Hz 44 mA psychomotor test is a long duration (3 s) and low frequency (6 Hz). Compounds active in the MES Identification Phase tests are screened for their ability to block seizures induced in the 6 Hz psychomotor focal seizures animal model of epilepsy. The compounds effective in the 6-Hz model during the Identification phase become candidates for advanced screening in the Differentiation Phase.

### Example 2

The PTZ seizure model may be used to evaluate the novel N-benzamide enaminone compounds of the invention.

In vivo target validation studies using the pentylenetrazole (PTZ) zebrafish model.

A pentylenetetrazol (PTZ) seizure model was previously described by Mussulini et al, using adult zebrafish. See Mussulini, B. H. M, et al. Seizures induced by pentylenetetrazole in the adult zebrafish: a detailed behavioral characterization. PloS one 2013, 8 (1), e54515-e54515. The PTZ seizure model may be used to validate the compounds of the invention using the following general method:

The test zebrafish is immersed in varying concentrations of PTZ (e.g., 2.5 mM-7.5 mM). Behavioral activity is monitored e.g., with video recording software for a 20-minute trial period, and compared to the control group (without PTZ exposure). See Cachat, et al. Measuring behavioral and endocrine responses to novelty stress in adult zebrafish. Nature Protocols 2010, 5 (11), 1786-1799.

The observation arena is divided equally into a virtual lower and upper zone using the software and the following endpoints are determined when the fish were moving; a) distanced traveled (cm) b) average velocity cm/s c) time spent in upper zone and d) time spent in lower zones of the observation tank.

### Example 3

### PGP Assay Analysis

The PGP-Glo™ Assay detects the effects of compounds on recombinant human Pgp in a cell membrane fraction. See Amaye et. Al. Int. J. Environ. Res. Public Health 2018, 15, 1784. The assay relies on the ATP dependence of the light-generating reaction of firefly luciferase. ATP is first incubated with Pgp; then the Pgp ATPase reaction is stopped, and the remaining unmetabolized ATP is detected as a luciferase-generated luminescent signal.

PGP-dependent decrease in luminescence reflect ATP consumption by PGP; thus the greater the decrease in signal, the higher the Pgp activity. Accordingly, samples containing compounds that stimulate the Pgp ATPase will have significantly lower signals than untreated samples.

This assay system is valuable for screening drugs and new chemical entities for their capacity to modulate P-glycoprotein ATPase activity. See FIG. 6 and Table 1.

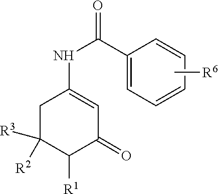

TABLE 1
Chemical Properties Fluorinated N-benzamide Enaminones

| | | | | | Yield | |
|---|---|---|---|---|---|---|
| Compound | R₁ | R₂ | R₃ | R₆ | (%) | MP ° C. |
| 12a | H | H | CH₃ | H | 18 | 151-154 |
| 12b | H | H | CH₃ | 4-CF₃ | 48 | 202-203 |
| 12c | H | H | CH₃ | 2-F, 4-CF₃ | 33 | 169-170 |
| 12d | H | H | CH₃ | 2-CF₃ | 21 | 180-181 |
| 12e | H | H | CH₃ | 3-CF₃ | 42 | 181-182 |
| 12f | H | H | CH₃ | 2-F | 25 | 126.7-127.7 |
| 12g | H | H | CH₃ | 4-F | 21 | 172.6-173.6 |
| 12h | H | H | CH₃ | 4-F, 2-CF₃ | 18.8 | 141-143 |
| 12i | H | H | CH₃ | 2-OCF₃ | 34 | 141-143 |
| 12j | H | H | CH₃ | 3-OCF₃ | 10 | 153-154 |
| 12k | H | H | CH₃ | 3,5-CF₃ | 34 | 185-187 |
| 12l | H | H | CF₃ | 4-CF₃ | 54.7 | 206.8-208 |
| 12m | H | H | CF₃ | 2-F, 4-CF₃ | 42.2 | 185.6-186.9 |
| 12n | H | H | CF₃ | 2-CF₃ | 19 | 148.9-150 |
| 12o | H | H | CF₃ | 2-F | 28 | 126.5-128.3 |
| 12p | H | H | CF₃ | 4-F | 32.1 | 212-214 |
| 12q | H | H | CF₃ | 4-F, 2-CF₃ | 5 | 175-177 |
| 12r | H | CH₃ | CH₃ | 2-OCF₃ | 42.3 | 153-155 |
| 12s | H | CH₃ | CH₃ | 3,5-CF₃ | 14 | 149-150 |
| 12t | H | H | CH(CH₃)₂ | 4-CF₃ | 52.9 | 194.5-195.8 |

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A compound of formula (II): 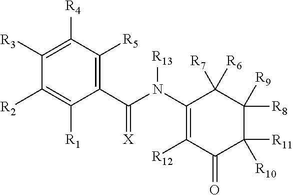
or a pharmaceutically acceptable salt thereof,
wherein
R₁ is —H, —OH, —CN, substituted or unsubstituted alkyl, substituted or unsubstituted alkenyl, substituted or unsubstituted alkynyl, halo, substituted or unsubstituted aryl, substituted or unsubstituted aryloxy, or substituted or unsubstituted alkyloxy;
each of R₂, R₃, R₄, or R₅ is independently selected from —H, —OH, —CN, substituted or unsubstituted alkyl, substituted or unsubstituted alkenyl, substituted or unsubstituted alkynyl, halo, substituted or unsubstituted aryl, substituted or unsubstituted aryloxy, OCF₃, SCF₃, NO₂, or substituted or unsubstituted alkyloxy;
each of R₆, R₇, R₈, R₉, R₁₀, or Ru is independently selected from —H, —OH, —CN, substituted or unsubstituted alkyl, substituted or unsubstituted alkenyl, substituted or unsubstituted alkynyl, halo, substituted or unsubstituted aryl, substituted or unsubstituted aryloxy, or substituted or unsubstituted alkyloxy;
R₁₂ is —H or substituted or unsubstituted alkyl;
X is O, S, or —NR₁₃; and
R₁₃ is independently selected from —H or substituted or unsubstituted alkyl,
wherein at least one of R₁-R₅ is —F, a substituted alkyl comprising one or more —F groups, or a substituted alkyloxy comprising one or more —F groups, and
wherein at least one of R₇-R₁₂ is —F, a substituted alkyl comprising one or more —F groups, or a substituted alkyloxy comprising one or more —F groups, and
wherein R₃ and Ra/R₉ are not identical.

2. A compound of formula (III): 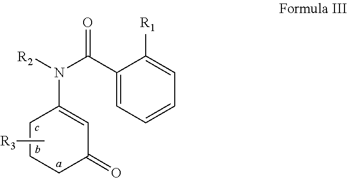
wherein
R₁ is a halogen or alkyl substituted with 1-3 halogen groups;
R₂ is hydrogen or C₁-C₃ alkyl;
R₃ is a hydroxyl, a halogen, cyano, amino, nitro, unsubstituted alkyl, or alkyl substituted with 1-3 halogen groups;
or a pharmaceutically acceptable salt, hydrate, solvate, enantiomer, diastereomer, racemate or mixture of stereoisomers thereof.

3. The compound of claim 2, wherein R₁ is a halogen.

4. The compound of claim 3, wherein halogen is F or Cl.

5. The compound of claim 3, wherein halogen is F.

6. The compound of claim 2, wherein Riis halogen and R₃ is substituted alkyl with 1-3 halogen groups.

7. (canceled)

8. (canceled)

9. (canceled)

10. The compound of claim 2, wherein R₁ is —F and R₃ is alkyl substituted with 1-3 halogen groups.

11. The compound of claim 2, wherein R₁ is —F and R₃ is —CF₃.

12. The compound of claim 2, wherein R₂ is H.

13. The compound of claim 2, wherein R₂ is —CH₃.

14. The compound of claim 2, wherein R₁ is —F, R₂ is —H, and R₃ is —CF₃.

15. The compound of claim 2, wherein R₃ is at position b.

16. The compound of claim 2, wherein R₁ is halo, R₃ is CF₃, and R₃ is at position b.

17. The compound of claim 2, wherein the compound has the following structure:

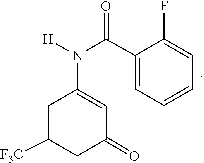 [SMILES: O=C1C=C(NC(=O)c2ccccc2F)CC(C(F)(F)F)C1]

18. A pharmaceutical composition comprising a compound according to formula (III): 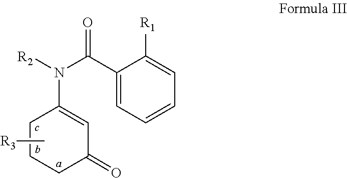
wherein
R₁ is a halogen or alkyl substituted with 1-3 halogen groups;
R₂ is hydrogen or C₁-C₃ alkyl;
R₃ is a hydroxyl, a halogen, cyano, amino, nitro, unsubstituted alkyl, or alkyl substituted with 1-3 halogen groups;
and a pharmaceutically acceptable carrier or excipient.

19. A The pharmaceutical composition of claim 18, wherein said compound of formula (III) is:

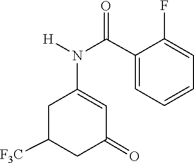 [SMILES: O=C1C=C(NC(=O)c2ccccc2F)CC(C(F)(F)F)C1]

20. A method of reducing the occurrence or severity of a seizure in a subject, or treating a subject at risk of having a seizure, said method comprising administering to said subject a pharmaceutical composition of claim 18.

21. (canceled)
